(19) United States
(12) Patent Application Publication
(10) Pub. No.: US 2020/0222410 A1
(43) Pub. Date: Jul. 16, 2020

(54) NUCLEOCYTOPLASMIC REGULATOR OF AUTOPHAGY-ASSOCIATED TRANSCRIPTION FACTORS

(71) Applicant: BROWN UNIVERSITY, Providence, RI (US)

(72) Inventor: Louis LAPIERRE, Barrington, RI (US)

(21) Appl. No.: 16/604,931

(22) PCT Filed: Apr. 13, 2018
(86) PCT No.: PCT/US2018/027640

§ 371 Date: Oct. 11, 2019

Related U.S. Application Data
(60) Provisional application No. 62/485,351, filed on Apr. 13, 2017.

(51) Int. Cl. A61K 31/52; A01K 67/033; C12N 15/113; A61P 35/00

(52) U.S. Cl. CPC A61K 31/52; A01K 67/0336; A01K 2227/703; A61P 35/00; C12N 15/113

(57) ABSTRACT

Provided herein, are compositions and methods of treatment for neurodegenerative diseases, such as neurodegenerative diseases associated with aging and methods for increasing longevity by inhibiting the expression of the protein exportin-1 (XPO1, CRM-1 or karyopherin) or a fragment thereof.

### CROSS-REFERENCE TO RELATED APPLICATIONS

This application claims priority to U.S. Provisional Patent Application No. 62/485,351, filed Apr. 13, 2018, the disclosure of which is incorporated by reference herein in its entirety.

### GOVERNMENT INTEREST

The invention was made with government support under R01 AG051810 and R00 AG042494 awarded by the National Institutes of Health. The government has certain rights in the invention.

### SEQUENCE LISTING

Incorporated by reference in its entirety is a computer-readable nucleotide/amino acid sequence listing submitted concurrently herewith and identified as follows: 45,056 byte text file named “35947-016001WO_Sequence_Listing_ST25.txt” created on Apr. 13, 2018.

### FIELD OF INVENTION

This invention relates, inter alia, to the identification of novel activators of autophagy to prevent proteostatic decline associated with neurodegenerative diseases and other aging-related disorders of the nervous system.

### BACKGROUND

Autophagy is a conserved cellular mechanism required for longevity across phyla (Lapierre, L. R., Kumsta, C., Sandri, M., Ballabio, A. & Hansen, M. Transcriptional and epigenetic regulation of autophagy in aging. Autophagy 11, 867-880, (2015)). Many different age-related diseases, including neurodegenerative diseases, are characterized by autophagic and lysosomal dysfunctions, which result in the accumulation of aberrant organelles and aggregates (Wong, E. & Cuervo, A. M. Autophagy gone awry in neurodegenerative diseases. Nat Neurosci 13, 805-811, (2010)). Therefore, to prevent the onset of these age-related diseases, the search for enhancers of autophagy is a priority in the scientific community.

### SUMMARY

The invention disclosed herein provides genetic and pharmacological regulators of the nucleocytoplasmic partitioning of HLH-30/TFEB and autophagy for the prevention of proteostatic decline associated with the development of neurodegeneration as well as methods for identifying the same.

Accordingly, in some aspects, provided herein are methods for treating a neurodegenerative disease in an individual comprising administering an inhibitor of exportin-1 (XPO1) to the individual. The neurodegenerative disease can be a disease associated with aging or it can be a disease selected from the group consisting of Alzheimer's disease, Amyotrophic lateral sclerosis (ALS), neurodegeneration in adult cases of Down's syndrome, Dementia puglistica, Pick's disease, Guam parkinsonism dementia complex, Fronto-temporal dementia, Cortico-Basal Degeneration, Pallido-Pontal-Nigral Degeneration, Progressive Nuclear Palsy, Parkinsonism of Chromosome 17 (FTDP-17), Parkinson's disease, Dementia with Lewy bodies, Huntington's disease, Multiple System Atrophy, fatty liver disease (liver steatosis), a1-anti-trypsin deficiency, muscle diseases, sporadic inclusion body myositis, limb girdle muscular dystrophy type 2B, and Miyoshi myopathy. In one embodiment, the neurodegenerative disease comprises Alzheimer's disease and administration of the inhibitor of XPO1 results in increased clearance of Aβ42 (such as any of about 10%, 20%, 30%, 40%, 50%, 60%, 70%, 80%, 90%, or 100% increased clearance of Aβ42). In further embodiments, administration of the inhibitor of XPO1 results in decreased formation of Huntington's disease-like polyQ-containing protein aggregates. In other embodiments, the neurodegenerative disease comprises Huntington's disease and administration of the inhibitor of XPO1 results in increased clearance of poly-glutamine(Q) protein (e.g., Q35 or Q40) (such as any of about 10%, 20%, 30%, 40%, 50%, 60%, 70%, 80%, 90%, or 100% increased clearance of Q35 and/or Q40). In another embodiment, the individual has not been diagnosed with cancer.

Further, administration of the inhibitor of XPO1 can result in the accumulation of autophagy-associated transcription factors in the nuclei of neurons and/or neural-related cells in the individual. In some embodiments, the autophagy-associated transcription factor comprises Transcription factor EB (TFEB). In other embodiments, administration of the inhibitor of XPO1 results in increased expression (such as any of about 10%, 20%, 30%, 40%, 50%, 60%, 70%, 80%, 90%, or 100% increased expression) of a gene encoding one or more of TFEB, Sequestosome-1 protein SQSTM1 p62 (p62), Microtubule-associated proteins 1A/1B light chain 3A (LC3), Forkhead box protein O (FOXO) or Arylsulfatase A (ARSA) polypeptides in neurons and/or neural-related cells in the individual. In yet additional embodiments, administration of the inhibitor of XPO1 results in increased autophagic flux (such as any of about 10%, 20%, 30%, 40%, 50%, 60%, 70%, 80%, 90%, or 100% increased autophagic flux) in neurons and/or neural-related cells in the individual. The inhibitor of XPO1 can be one or more agents selected from the group consisting of a small molecule chemical compound, an antisense oligonucleotide, a siRNA, a non-antibody peptide, or an antibody or functional fragment thereof. The small molecule chemical compound can be an inhibitor of nuclear export, such as Selenixor (KPT-330), KPT-276, KPT-185, and KPT-335 (Verdinexor). The small molecule inhibitor of nuclear export can be administered in doses of 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 15 μM, 20 μM, 25 μM, 30 μM, 35 μM, 40 μM, 45 μM, 50 μM, 60 μM, 70 μM, 80 μM, 90 μM, or 100 μM or greater amounts, inclusive of all ranges and values falling in between these concentrations. In one embodiment, the inhibitor of XPO1 comprises Selenixor (KPT-330) (such as a therapeutically effective amount of Selenixor).

In further aspects, provided herein are methods for identifying one or more genes regulated by HLH30/Transcription factor EB (TFEB) and/or DAF-16/Forkhead box protein O (FOXO), or an orthologue thereof, in a cell comprising contacting the cell with an inhibitor of exportin-1 (XPO1) or an orthologue thereof and identifying one or more genes whose expression changes following contact with the inhibitor of XPO1, wherein the expression of said one or more genes changes due to increased expression or nuclear localization of HLH30/TFEB and/or DAF-16/FOXO following inhibition of XPO1. In some embodiments, the cell is a mammalian cell, an insect cell, a fish cell, or a nematode cell (such as a C. elegans cell). The mammalian cell can comprise a neural cell, a dermal cell such as a hypodermis cell, an intestinal cell, or a muscle cell. The inhibitor of XPO1 can be one or more agents selected from the group consisting of a small molecule chemical compound, an antisense oligonucleotide, a siRNA, a non-antibody peptide, or an antibody or functional fragment thereof. The small molecule chemical compound can be an inhibitor of nuclear export, such as Selenixor (KPT-330), KPT-276, KPT-185, and KPT-335 (Verdinexor). The small molecule inhibitor of nuclear export can be administered in doses of 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 15 μM, 20 μM, 25 μM, 30 μM, 35 μM, 40 μM, 45 μM, 50 μM, 60 μM, 70 μM, 80 μM, 90 μM, or 100 μM or greater amounts, inclusive of all ranges and values falling in between these concentrations. In some embodiments, the cell has been engineered to express fluorescently-tagged HLH30/TFEB and/or DAF-16/FOXO. In additional embodiments, changes in gene expression are identified by one or more of qPCR, ChIP qPCR, microarray, northern blot, or immunoblot.

In yet other embodiments, provided herein are methods for increasing the longevity of a cell comprising contacting the cell with an inhibitor of exportin-1 (XPO1). The cell can be, without limitation, a mammalian cell, an insect cell, a fish cell, or a nematode cell (such as a C. elegans cell). The inhibitor of XPO1 can be one or more agents selected from the group consisting of a small molecule chemical compound, an antisense oligonucleotide, a siRNA, a non-antibody peptide, or an antibody or functional fragment thereof. The small molecule chemical compound can be an inhibitor of nuclear export, such as Selenixor (KPT-330), KPT-276, KPT-185, and KPT-335 (Verdinexor). The small molecule inhibitor of nuclear export can be administered in doses of 500 nM, 600 nM, 700 nM, 800 nM, 900 nM, 1 μM, 2 μM, 4 μM, 5 μM, 6 μM, 7 μM, 8 μM, 9 μM, 10 μM, 15 μM, 20 μM, 25 μM, 30 μM, 35 μM, 40 μM, 45 μM, 50 μM, 60 μM, 70 μM, 80 μM, 90 μM, or 100 μM or greater amounts, inclusive of all ranges and values falling in between these concentrations.

Each of the aspects and embodiments described herein are capable of being used together, unless excluded either explicitly or clearly from the context of the embodiment or aspect.

Throughout this specification, various patents, patent applications and other types of publications (e.g., journal articles, electronic database entries, etc.) are referenced. The disclosure of all patents, patent applications, and other publications cited herein are hereby incorporated by reference in their entirety for all purposes.

### BRIEF DESCRIPTION OF THE DRAWINGS

FIG. 1A is a schematic depicting the autophagic process in cells.

FIG. 2A-FIG. 2H depict XPO-1 modulation of the nuclear localization of HLH-30/TFEB and autophagy. FIG. 2A is a series of fluorescent micrographs depicting worms expressing HLH-30::GFP grown during development or during adulthood for 72 hours on control bacteria or bacteria expressing RNAi against xpo-1 (100× magnification). FIG. 2B is a bar graph showing expression levels of xpo-1, lgg-1, lgg-2, sqst-1 and hlh-30 measured by qPCR in animals fed control bacteria or bacteria expressing RNAi against xpo-1 from day 1 to day 5 of adulthood. *: P<0.05, **P<0.01, N=4, t-test. Autophagosome and autolysosome formation were measured in hypodermal seam cells (FIG. 2C; left panels=control; right panels=xpo-1 RNAi) and pharynx (FIG. 2D; top panel and third panel from top=control; second panel from top and bottom panel=xpo-1 RNAi) of wild-type and hlh-30 (tm1978) animals expressing tandem autophagy reporter mCherry::GFP::LGG-1 and incubated with control bacteria or bacteria expressing RNAi against xpo-1 for 48 hours during early adulthood. *: P<0.05, **P<0.01, N=8, t-test. FIG. 2E is a line graph showing the effect of xpo-1 silencing (dark line=control; light line=xpo-1 RNAi) during 7 days of adulthood on heat resistance P<0.05, N=100, Mantel-Cox log rank. Accumulation of Aβ42 (FIG. 2F) and Q35:: GFP punctae (FIG. 2G and FIG. 2H) were measured in transgenic animals fed control bacteria or bacteria expressing RNAi against xpo-1 during 5 days of adulthood. *: P<0.05, Q35::GFP (N=5), Aβ42 (N=100), t-test.

FIG. 3A-FIG. 3I depict that silencing xpo-1 extends lifespan in C. elegans. Lifespan analyses of wild-type (WT; FIG. 3A), hlh-30 (tm1978; FIG. 3B), daf-16 (mu86; FIG. 3C), atg-7 (bp411; FIG. 3D), atg-18 (gk378; FIG. 3E), daf-36 (k114; FIG. 3F), eat-2 (ad1116; FIG. 3G), glp-1 (e2144; FIG. 3H), and rsks-1 (sv31; FIG. 3I) fed control bacteria or bacteria expressing dsRNA against xpo-1 from day 1 of adulthood. N=100, Mantel-Cox log rank. See Table 3 for statistical analyses and repeats. For all figures, the dark line represents the control condition while the lighter line is the RNAi-treatment condition.

FIG. 4A-FIG. 4F depict that pharmacological inhibition of XPO-1/Embargoed promotes autophagy and longevity. FIG. 4A is a series of fluorescent micrographs showing day 1 animals expressing HLH-30::GFP that were fed with OP50 E. coli bacteria with DMSO (0.1%) or KPT-330 at concentrations of 25, 50 or 100 μM. (100× magnification). FIG. 4B is a graph depicting lifespan analysis of wild-type animals fed bacteria with DMSO 0.1% (black line) or KPT-330 at (25 μM (light grey line), 50 μM (grey line) or 100 μM (dark grey line)) throughout lifespan. Autophagosome and autolysosome were quantified in the pharynx (FIG. 4C) and in hypodermal seam cells (FIG. 4D; top panel=control; middle panel=50 μM KPT-330; bottom panel=100 μM KPT-330) of animals expressing tandem autophagy reporter mCherry::GFP::LGG-1 and incubated with bacteria with DMSO (0.1%) or KPT-330 at concentrations of 50 or 100 μM from day 1 to day 3 of adulthood. FIG. 4E is a line graph depicting the results of a survival assay under heat stress of animals fed bacteria with DMSO (0.1%; dark line) or KPT-330 (light line) at 100 μM from day 1 to day 5 of adulthood P<0.05, N˜100, Mantel-Cox log rank. FIG. 4F is a line graph depicting the results of a Lifespan analysis of ALS model in flies (dsodH71Y) fed food with DMSO (0.1%; dark line) or KPT-330 (light line) at 100 μM. P<0.05, N>300, Mantel-Cox log rank. See Table 4 for statistical analyses and repeats.

FIG. 5A-FIG. 5F depict nuclear enrichment of TFEB and autophagy are stimulated by XPO1 inhibition. FIG. 5A is a series of fluorescent micrographs showing TFEB-GFP expressing HeLa cells that were incubated in a medium containing vehicle or compounds (Torin 1 5 μM, KPTs 1 μM) for 6 hours and fixed cells were imaged. Scale bar 20 mm FIG. 5B is a bar graph showing the percentage of cells with TFEB nuclear localization was quantified from four independent experiments as described in FIG. 5A (from left to right, control, Torin 1, KPT-330, KPT-276, KPT-185, and KPT-335). FIG. 5C is a series of fluorescent micrographs showing HeLa cells that were grown in medium containing vehicle or compounds for 6 hours and lysosomes were visualized with Lysotracker Red and signal intensities were quantified as shown in the bar graph depicted in FIG. 5D. *: Image taken at half the exposure for representational purposes. Scale bar 50 mm (top left panel=control; top right panel=Torin 1; middle left panel=KPT-330; middle right panel=KPT-276; bottom left panel=KPT-185; bottom right panel=KPT-335). FIG. 5E is an image showing HeLa cells grown in a medium containing DMSO (0.1%) or compounds for 24 hours and proteins were visualized by immunoblotting. FIG. 5F is a bar graph showing levels of LC3 I and II quantified by densitometry (from left to right, control, Torin 1, KPT-330, KPT-276, KPT-185, and KPT-335), One-way ANOVA, *: P<0.05, **: P<0.01, ***: P<0.001, a: P=0.054, b: P=0.073. Images and blots are representative of three independent experiments.

FIG. 6 depicts an amino acid sequence alignment showing that XPO-1/XPO1 is a conserved nuclear export protein. A multiple sequence alignment of Caenorhabditis elegans (XPO-1), Drosophila melanogaster (Embargoed) and Homo sapiens (CRM-1/XPO1) performed with T-Coffee and visualized with BoxShade is shown.

FIG. 7A-FIG. 7F depict that longevity associated with xpo-1 inhibition mimics long-lived models. FIG. 7A is a graph showing that synchronized wild-type eggs fed throughout lifespan control bacteria (dark line) or bacteria expressing RNAi against xpo-1 (light line) (See Table 3 for details). FIG. 7B is a fluorescent micrograph showing day 1 animals expressing DAF-16::GFP (CF1934) and exposed to control RNAi (left) or RNAi against xpo-1 (right). FIG. 7C is a bar graph showing levels of xpo-1 mRNA measured by qPCR in wild-type (WT) at non-permissive and permissive temperature (25° C. and 20° C., respectively), glp-1(e2144) at non-permissive temperature, eat-2(ad1116) and rsks-1(sv31). N=4, *: P<0.05, t-test. FIG. 7D is a light micrograph showing Oil-Red-O staining of wild-type animals fed for 7 days control bacteria or bacteria expressing xpo-1 RNAi. FIG. 7E is a bar graph showing levels of lysosomal acid lipase genes lipl-1, lipl-2, lipl-3 and lipl-4 in Day 5 wild-type animals fed control bacteria (dark bars) or bacteria expressing RNAi against xpo-1 (light bars) since Day 1 of adulthood. N=4, *: P<0.05, t-test. FIG. 7F is a bar graph showing xpo-1, lgg-1, lgg-2, sqst-1 and hlh-30 mRNA levels quantified by qPCR in glp-1(e2144) animals fed control bacteria (dark bars) or bacteria expressing RNAi against xpo-1 from Day 1 to Day 5 of adulthood (light bars). N=4, *: P<0.05, t-test.

FIG. 8A-FIG. 8E depicts pharmacological inhibition of XPO-1 increases lifespan. FIG. 8A is a series of fluorescent micrographs showing animals expressing HLH-30::GFP that were fed OP50 E. coli bacteria containing vehicle (DMSO 0.1%), KPT-330 (1, 10 and 25 μM) or KPT-276 (25 μM) for 48 hours (100× magnification). FIG. 8B is a line graph showing the results of a lifespan analysis of worms fed OP50 E. coli bacteria containing vehicle (DMSO 0.1%; dark line) or KPT-276 (25 μM; light line) during adulthood (see Table 4 for details). FIG. 8C is a bar graph and fluorescent micrograph depicting quantification of Q40::YFP aggregates in day 5 animals fed control bacteria or bacteria containing 100 μM KPT-330 or 25 μM KPT-276. Micrographs included below histogram (100× magnification) N=5, *: P<0.05, t-test. Lifespan analysis of H71Y male (FIG. 8D; control=dark line; KPT-330=light line) and female (FIG. 8E; control=dark line; KPT-330=light line) flies (see Table 4 for details).

FIG. 9A-FIG. 9E depicts XPO1 inhibition and silencing enhances TFEB nuclear localization and lysosome biogenesis in a TOR-independent manner. FIG. 9A is a fluorescent micrograph showing measurement of GFP levels (Scale bar=20 μm) and lysotracker (Scale bar on image=50μ) staining in HeLa cells expressing TFEB-GFP that were incubated for 48 with Control RNAi or RNAi against XPO1. FIG. 9B is a series of fluorescent micrographs showing HeLa cells expressing TFEB-GFP that were incubated for 6 hours with 5 μM of GSK-3β inhibitor VIII or 10 nM of Leptomycin B. FIG. 9B is a series of fluorescent micrographs showing Hela cells that were incubated for 48 with Control RNAi or RNAi against TFEB and then subjected to 6 hours of DMSO 0.1% (Control), 2 μM of Torin 1 or 1 μM of KPTs. Lysotracker staining was performed (Scale bar=50 μm). A separate set of HeLa cells expressing TFEB-GFP were used to test the efficiency of TFEB RNAi (see inset). Lysotracker signal was quantified and the percentage of signal in siTFEB-treated cells vs control is shown. FIG. 9D is a bar graph showing the ratio of LC3II/LC3I quantified from densitometric analyses. FIG. 9E is a bar graph showing densitometric quantification of phospho-mTOR (p-mTOR) immunoblotting and associated independent repeats. Phosphorylated levels of mTOR were normalized with the immunoblots of the corresponding protein.

### DETAILED DESCRIPTION

The invention described herein provides treatments for neurodegenerative diseases and methods for increasing longevity via inhibiting the expression or activity of the protein exportin-1 (XPO1, CRM-1 or karyopherin). XPO1 is involved in recognizing and transporting proteins containing leucine-rich nuclear export sequences. Reversible inhibitors of XPO1, such as Selinexor, show selectivity and bioavailability. Selinexor has the ability to cross the blood-brain barrier, which is especially important in the context of targeting neurodegenerative diseases.

### Autophagy and Regulation thereof

Macroautophagy (referred to autophagy hereafter) consists of the bulk sequestration of intracellular material into a vesicle called the autophagosome, which eventually fuses to the lysosome for degradation.

A transcription factor called TFEB was found to preferentially enhance the expression of autophagy and lysosomal genes, indicating autophagy can be regulated transcriptionally (Settembre, C. et al. TFEB links autophagy to lysosomal biogenesis. Science 332, 1429-1433, (2011); Sardiello, M. et al. A gene network regulating lysosomal biogenesis and function. Science 325, 473-477, (2009)). The nematode C. elegans possesses an ortholog of TFEB called HLH-30 that also modulates autophagy and lifespan in multiple longevity models (Lapierre, L. R. et al. The TFEB orthologue HLH-30 regulates autophagy and modulates longevity in Caenorhabditis elegans. Nat Commun 4, 2267, (2013)). The nuclear localization of HLH-30/TFEB is negatively regulated by a nutrient sensor called the mechanistic target of Rapamycin (mTOR) (Settembre, C. et al. TFEB controls cellular lipid metabolism through a starvation-induced autoregulatory loop. Nat Cell Biol 15, (2013); O'Rourke, E. J. & Ruvkun, G. MXL-3 and HLH-30 transcriptionally link lipolysis and autophagy to nutrient availability. Nat Cell Biol 15, 668-676, (2013)). While Rapamycin activates autophagy, associated negative and adverse side effects of mTOR inhibition (Kennedy, B. K. & Lamming, D. W. The Mechanistic Target of Rapamycin: The Grand ConducTOR of Metabolism and Aging. Cell Metab 23, 990-1003, (2016)) has weakened this potential therapeutic approach, and compelled the scientific community to seek more specific activators of autophagy.

As described in more detail herein, XPO1 inhibition results in increased nuclear expression and accumulation of gene products associated with autophagy, specifically, increased expression and nuclear localization of autophagy-associated transcription factors and/or polypeptides in the nuclei of neurons and/or neural-related cells. Without being bound to theory, since many different age-related diseases, including neurodegenerative diseases, are characterized by autophagic and lysosomal dysfunction (often resulting in the accumulation of aberrant organelles and aggregates) increasing the availability and expression autophagy-associated transcription factors and/or polypeptides in the nuclei of neural cells restores proper autophagic flux and results in the improvement of neurodegenerative disease symptoms.

Also provided herein are methods for identifying one or more genes regulated by HLH30/Transcription factor EB (TFEB) and/or DAF-16/Forkhead box protein O (FOXO), or an orthologue thereof, in a cell. These factors have been shown to modulate autophagy and lifespan in multiple longevity models 5 and inhibition of XPO1 is shown herein to increase their expression and accumulation in the nuclei of neural tissue. Thus, the present invention provides methods for identifying other genes capable of affecting autophagic flux by observing changes in gene expression in cells following inhibition of XPO1.

### I. Definitions

The term “neurodegenerative disease” as used herein refers to central nervous system disorders characterized by gradual and progressive loss of neural tissue and/or neural tissue function, with typically reduced neurological function as a result of a gradual and progressive loss of neural tissue. In some embodiments, the neurodegenerative diseases amenable to prevention and/or treatment using the methods as described herein are neurodegenerative diseases associated with aging or senescence in an individual.

As used herein, the term “protein” includes polypeptides, peptides, fragments of polypeptides, and fusion polypeptides.

As used herein, a “nucleic acid” refers to two or more deoxyribonucleotides and/or ribonucleotides covalently joined together in either single or double-stranded form.

An “individual” or a “subject” can be a vertebrate, a mammal, or a human. In some embodiments, a subject can be a laboratory model organism, such as (without limitation) a nematode (e.g. C. elegans), a fish (e.g., zebrafish), or an insect (e.g. D. melanogaster). Mammals include, but are not limited to, farm animals, sport animals, companion animals such as pets, primates, mice and rats. In one aspect, a subject is a human. In some embodiments of the invention disclosed herein, the individual has been diagnosed with a neurodegenerative disease, such as a neurodegenerative disease associated with aging. For example, the individual in some embodiments has been diagnosed with Alzheimer's disease based on, without limitation, the NINCDS-ADRDA Alzheimer's Criteria for diagnosis which requires that the presence of cognitive impairment and a suspected dementia syndrome be confirmed by neuropsychological testing for a clinical diagnosis of possible or probable AD. In other embodiments, the individual has been diagnosed with ALS based on, without limitation, the progressive worsening of symptoms such as muscle weakness, atrophy of muscles, hyperreflexia, and spasticity. The individual in yet other embodiments has been diagnosed with HD based on, without limitation, the presence of an expanded copy of the trinucleotide repeat in the HTT gene that causes the disease. In other embodiments, the individual has not been diagnosed with nor is suspected to have cancer. In further embodiments, the individual is over 60 years of age such as over 65, 70, 75, 80, 85, 90, 95, or 100 years of age).

The terms “treating” and “treatment” as used herein refer to the administration of an agent or formulation to a clinically symptomatic individual afflicted with an adverse condition, disorder, or disease, so as to effect a reduction in severity and/or frequency of symptoms, eliminate the symptoms and/or their underlying cause, and/or facilitate improvement or remediation of damage.

An “effective amount” or “therapeutically effective amount” refers to an amount of therapeutic compound, such as an oligonucleotide, small molecule, antibody, or any other anticancer therapy, administered to a subject, either as a single dose or as part of a series of doses, which is effective to produce a desired therapeutic effect.

The phrase “inhibiting the activity of a gene, protein, or fragment thereof,” as used herein, means inhibiting one or more or all of the biological and/or biochemical functions of a polypeptide (such as XPO1) or a gene encoding that polypeptide,

The phrase “inhibiting the expression of polypeptide,” as used herein, means inhibiting the expression of a gene (such as a gene encoding XPO1) at the level of DNA transcription into RNA or RNA translation into protein, thereby resulting in decreased or no RNA and/or protein in a cell. In some embodiments, inhibiting the expression of one or more polypeptides encompasses manipulating a cell to cause proteolytic degradation of one or more protein(s). In some embodiments, inhibiting the expression of one or polypeptides encompasses manipulating a cell to cause degradation of one or more RNA(s).

“Purified protein” as used herein means the protein or fragment or functional fragment (e.g. domains) is sufficiently free of contaminants or cell components with which the protein normally occurs to distinguish the protein from the contaminants or cell components. It is not contemplated that “purified” necessitates having a preparation that is technically totally pure (homogeneous), but purified as used herein means the protein or polypeptide fragment is sufficiently separated from contaminants or cell components with which it normally occurs to provide the protein in a state where it can be used in an assay, such as immunoprecipitation or ELISA, or can be used as an agent in a therapeutic treatment.

“Purified nucleic acid” as used herein means the nucleic acid (such as an antisense oligonucleotide or an siRNA) is sufficiently free of contaminants or cell components with which the nucleic acid normally occurs to distinguish the nucleic acid from the contaminants or cell components. It is not contemplated that “purified” necessitates having a preparation that is technically totally pure (homogeneous), but purified as used herein means the nucleic acid is sufficiently separated from contaminants or cell components with which it normally occurs to provide the nucleic acid in a state where it can be used in an assay or can be used as an agent in a therapeutic treatment.

“Purified chemical compound” (such as a small molecule chemical compound) as used herein means the compound is sufficiently free of chemical contaminants resulting from its isolation or chemical synthesis to distinguish the chemical compound from the contaminants.

Unless defined otherwise herein, all technical and scientific terms used herein have the same meaning as commonly understood by one of ordinary skill in the art to which this invention pertains.

As used herein, the singular terms “a,” “an” and “the” include the plural reference unless the context clearly indicates otherwise,

### II. Methods of the Invention

Neurodegenerative diseases and neurodegenerative diseases associated with aging are characterized by a wide range of symptoms which vary in severity and range from individual to individual. For example, Alzheimer's disease is characterized by symptoms such as depression, aggression, impairment in short-term memory, impairment in intellectual ability, agitation, irritability and restlessness. A common feature of neurodegenerative disorders and the process of aging in animals is the progressive cell damage of neurons within the central nervous system (CNS) leading to loss of neuronal activity and cell death. This loss of activity has been correlated with adverse behavioral symptoms including memory loss and cognitive deficits. Therapeutic agents that have been developed to retard loss of neuronal activity either have toxic side effects or are prevented from reaching their target site because of their inability to cross the blood-brain barrier. The blood-brain barrier is a complex of morphological and enzymatic components that retards the passage of both large and charged small molecules thereby limiting access to cells of the brain.

### A. Methods for Treating Neurodegenerative Diseases

Provided herein are methods for treating a neurodegenerative disease in an individual by administering to the individual an inhibitor of exportin-1 (XPO1) or a functional domain thereof. In some embodiments, the individual is characterized or diagnosed as comprising a neurodegenerative disease. Examples of neurological diseases and neurodegenerative diseases and disorders include, but are not limited, to Alzheimer's disease (AD), amyotrophic lateral sclerosis (ALS), and Huntington's disease (HD). In other embodiments, the individual has not been diagnosed with nor is suspected to have cancer.

Alzheimer's disease (AD) is a progressive disease resulting in senile dementia. See generally Selkoe, TINS 16, 403-409 (1993); Hardy et al., WO 92/13069; Selkoe, J. Neuropathol. Exp. Neurol. 53, 438-447 (1994); Duff et al., Nature 373, 476-477 (1995); Games et al., Nature 373, 523 (1995). Broadly speaking the disease falls into two categories: late onset, which occurs in old age (65+ years) and early onset, which develops well before the senile period, i.e., between 35 and 60 years. In both types of disease, the pathology is the same but the β abnormalities tend to be more severe and widespread in cases beginning at an earlier age. The disease is characterized at the macroscopic level by significant brain shrinkage away from the cranial vault as seen in MRI images as a direct result of neuronal loss and by two types of macroscopic lesions in the brain, senile plaques and neurofibrillary tangles. Senile plaques are areas comprising disorganized neuronal processes up to 150 μm across and extracellular amyloid deposits, which are typically concentrated at the center and visible by microscopic analysis of sections of brain tissue. Neurofibrillary tangles are intracellular deposits of tau protein consisting of two filaments twisted about each other in pairs. The principal constituent of Alzheimer's plaques is a peptide termed Aβ or β-amyloid peptide. AP peptide is an internal fragment of 39-43 amino acids of a precursor protein termed amyloid precursor protein (APP). Several mutations within the APP protein have been correlated with the presence of Alzheimer's disease. Alzheimer's disease can be recognized and diagnosed based on characteristic dementia, as well as the presence of genetic risk factors known in the art. In addition, a number of diagnostic tests are available for identifying subjects who have Alzheimer's disease. These include measurement of CSF tau and Aβ342 levels. Elevated tau and increased Aβ342 levels signify the presence of Alzheimer's disease. Individuals suffering from Alzheimer's disease can also be diagnosed by MMSE or ADRDA criteria.

Amyotrophic lateral sclerosis (ALS), also known as Lou Gehrig's disease and motor neurone disease (MND), is a specific disease that causes the death of neurons which control voluntary muscles. ALS is characterized by stiff muscles, muscle twitching, and gradually worsening weakness due to muscles decreasing in size. This results in difficulty speaking, swallowing, and eventually breathing. Because symptoms of ALS can be similar to those of a wide variety of other, more treatable diseases or disorders, appropriate tests must be conducted to exclude the possibility of other conditions. One of these tests is electromyography (EMG), a special recording technique that detects electrical activity in muscles. Certain EMG findings can support the diagnosis of ALS. Another common test measures nerve conduction velocity (NCV). ALS must be differentiated from the “ALS mimic syndromes” which are unrelated disorders that may have a similar presentation and clinical features to ALS or its variants. Because of the prognosis carried by this diagnosis and the variety of diseases or disorders that can resemble ALS in the early stages of the disease, people with ALS symptoms should always obtain a specialist neurological opinion in order to rule out alternative diagnoses.

Huntington's disease (HD), also known as Huntington's chorea, is an inherited disorder that results in death of brain cells. The earliest symptoms are often subtle problems with mood or mental abilities. A general lack of coordination and an unsteady gait often follow. As the disease advances, uncoordinated, jerky body movements become more apparent. Physical abilities gradually worsen until coordinated movement becomes difficult and the person is unable to talk. Mental abilities generally decline into dementia. The specific symptoms vary somewhat between people. Symptoms usually begin between 30 and 50 years of age, but can start at any age. A physical examination, sometimes combined with a psychological examination, can determine whether the onset of the disease has begun. Excessive unintentional movements of any part of the body are often the reason for seeking medical consultation. If these are abrupt and have random timing and distribution, they suggest a diagnosis of HD. Cognitive or behavioral symptoms are rarely the first symptoms diagnosed; they are usually only recognized in hindsight or when they develop further. How far the disease has progressed can be measured using the unified Huntington's disease rating scale, which provides an overall rating system based on motor, behavioral, cognitive, and functional assessments. Medical imaging, such as computerized tomography (CT) and magnetic resonance imaging (MRI), can show atrophy of the caudate nuclei early in the disease, as seen in the illustration to the right, but these changes are not, by themselves, diagnostic of HD. Cerebral atrophy can be seen in the advanced stages of the disease. Functional neuroimaging techniques, such as functional magnetic resonance imaging (fMRI) and positron emission tomography (PET), can show changes in brain activity before the onset of physical symptoms, but they are experimental tools, and are not used clinically. Further, Because HD follows an autosomal dominant pattern of inheritance, there is a strong motivation for individuals who are at risk of inheriting it to seek a diagnosis based on widely available genetic tests.

Additional neurodegenerative diseases which are amenable for treatment using the inhibitors of XPO1 disclosed herein include, for example and without limitation, Parkinson's disease, vascular dementia, aging and mild-cognitive impairment, age-related memory impairment, agyrophilic grain dementia, Parkinsonism-dementia complex of Guam, auto-immune conditions (e.g. Guillain-Barre syndrome, Lupus), Biswanger's disease, brain and spinal tumors (including neurofibromatosis), cerebral amyloid angiopathies (Journal of Alzheimer's Disease vol 3, 65-73 (2001)), cerebral palsy, chronic fatigue syndrome, corticobasal degeneration, conditions due to developmental dysfunction of the CNS parenchyma, conditions due to developmental dysfunction of the cerebrovasculature, dementia—multi infarct, dementia—subcortical, dementia with Lewy bodies, dementia of human immunodeficiency virus (HIV), dementia lacking distinct histology, Dementia Pugilistica, diffies neurofibrillary tangles with calcification, diseases of the eye, ear and vestibular systems involving neurodegeneration (including macular degeneration and glaucoma), Down's syndrome, dyskinesias (Paroxysmal), dystonias, essential tremor, Fahr's syndrome, fronto-temporal dementia and Parkinsonism linked to chromosome 17 (FTDP-17), frontotemporal lobar degeneration, frontal lobe dementia, hepatic encephalopathy, hereditary spastic paraplegia, hydrocephalus, pseudotumor cerebri and other conditions involving CSF dysfunction, Gaucher's disease, Hallervorden-Spatz disease, Korsakoffs syndrome, mild cognitive impairment, monomeric amyotrophy, motor neuron diseases, multiple system atrophy, multiple sclerosis and other demyelinating conditions (eg leukodystrophies), myalgic encephalomyelitis, myoclonus, neurodegeneration induced by chemicals, drugs and toxins, neurological manifestations of AIDS including AIDS dementia, neurological/cognitive manifestations and consequences of bacterial and/or virus infections, including but not restricted to enteroviruses, Niemann-Pick disease, non-Guamanian motor neuron disease with neurofibrillary tangles, non-ketotic hyperglycinemia, olivo-ponto cerebellar atrophy, oculopharyugeal muscular dystrophy, neurological manifestations of Polio myelitis including non-paralytic polio and post-polio-syndrome, primary lateral sclerosis, prion diseases including Creutzfeldt-Jakob disease (including variant form), kuru, fatal familial insomnia, Gerstmann-Straussler-Scheinker disease and other transmissible spongiform encephalopathies, prion protein cerebral amyloid angiopathy, postencephalitic Parkinsonism, progressive muscular atrophy, progressive bulbar palsy, progressive subcortical gliosis, progressive supranuclear palsy, restless leg syndrome, Rett syndrome, Sandhoff disease, spasticity, sporadic fronto-temporal dementias, striatonigral degeneration, subacute sclerosing panencephalitis, sulphite oxidase deficiency, Sydenham's chorea, tangle only dementia, Tay-Sach's disease, Tourette's syndrome, vascular dementia, and Wilson disease.

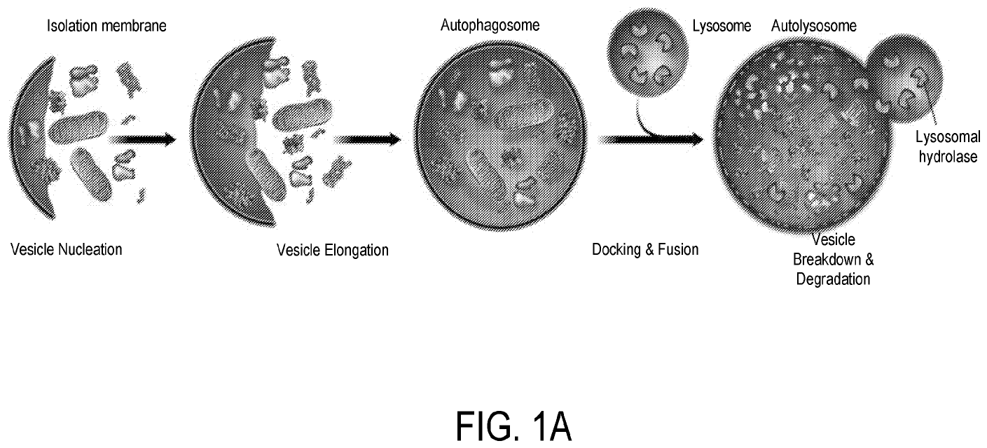

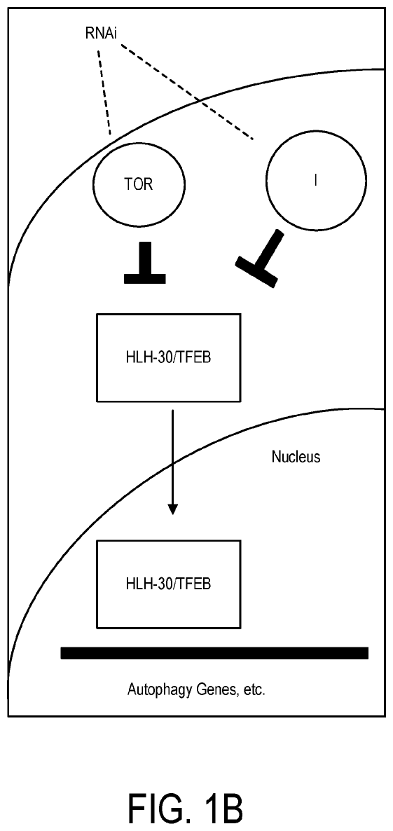

In some embodiments, administration of an XPO1 inhibitor to an individual in need thereof (such as an individual diagnosed with or characterized by having a neurodegenerative disease) results in increased autophagic flux in neurons or neural-related tissue or cells in the individual. “Autophagy” or “autophagic flux” as used herein means a catabolic process involving the degradation of a cell's own components through the lysosomal machinery. It is a tightly regulated process which plays a normal part in cell growth, development, and homeostasis, where it helps maintain a balance between the synthesis, degradation, and subsequent recycling of cellular products. It is a major mechanism by which a starving cell reallocates nutrients from unnecessary processes to more essential processes. A variety of autophagic processes exist, all sharing in common the degradation of intracellular components via the lysosome. The most well-known mechanism of autophagy involves the formation of a membrane around a targeted region of the cell, separating the contents from the rest of the cytoplasm. The resultant vesicle then fuses with a lysosome and subsequently degrades the contents (FIG. 1A). Autophagic flux can be measured by any number of means well-known in the art, including those described in Example 2.

In some embodiments, administration of an XPO1 inhibitor or an inhibitor of a fragment of XPO1 to an individual in need thereof (such as an individual diagnosed with or characterized by having a neurodegenerative disease) results in increased autophagic flux in neurons or neural-related tissue or cells in the individual by any of about 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% or more. The XPO1 inhibitor or an inhibitor of a fragment of XPO1 can be one or more agents selected from the group consisting of a small molecule chemical compound, an antisense oligonucleotide, a siRNA, a non-antibody peptide, or an antibody or functional fragment thereof.

Moreover, in further embodiments, administration of the inhibitor of XPO1 or an inhibitor of a fragment of XPO1 results in the accumulation of autophagy-associated transcription factors and/or polypeptides in the nuclei of neurons and/or neural-related cells in the individual. These autophagy-associated polypeptides transcription factors can include, without limitation, Transcription factor EB (TFEB; NCBI Reference Sequence: NM_001167827.2), Sequestosome-1 protein SQSTM-1, p62 (p62; NCBI Reference Sequence: NM_003900.4), Microtubule-associated proteins 1A/1B light chain 3A (LC3; NCBI Reference Sequence: NM_032514.3), a Forkhead box protein O (FOXO) or Arylsulfatase A (ARSA; NCBI Reference Sequence: NM_000487.5). For example, administration of the XPO1 inhibitor can result in any of about 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% or more increased accumulation of autophagy-associated transcription factors in the nuclei of neurons and/or neural-related cells in the individual relative to the accumulation of these polypeptides in the nuclei of neurons and/or neural-related cells of individuals who have not been administered an inhibitor of XPO1 or an inhibitor of a fragment of XPO1. Accumulation of one or more autophagy-associated transcription factors and/or polypeptides can be measured by any means known in the art, such as those described in Examples 1 and 4. The XPO1 inhibitor or an inhibitor of a fragment of XPO1 can be one or more agents selected from the group consisting of a small molecule chemical compound, an antisense oligonucleotide, a siRNA, a non-antibody peptide, or an antibody or functional fragment thereof.

Further, administration of the inhibitor of XPO1 or an inhibitor of a fragment of XPO1 to an individual diagnosed with a neurodegenerative disease can result in increased expression of one or more autophagy-associated transcription factors and/or polypeptides (e.g., any of TFEB, p62, or LC3), such as any of about 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% or more increased expression relative to the expression of these polypeptides in neurons and/or neural-related cells of individuals who have not been administered an inhibitor of XPO1 or an inhibitor of a fragment of XPO1. Expression of autophagy-associated transcription factors and/or polypeptides can be measured by any means known in the art, such as those described in Example 1. The XPO1 inhibitor or an inhibitor of a fragment of XPO1 can be one or more agents selected from the group consisting of a small molecule chemical compound, an antisense oligonucleotide, a siRNA, a non-antibody peptide, or an antibody or functional fragment thereof.

In other embodiments, the neurodegenerative disease is Alzheimer's disease and administration of the XPO1 inhibitor or an inhibitor of a fragment of XPO1 to an individual diagnosed with or thought to have Alzheimer's disease results in increased clearance of Aβ42 in the neurons or neural-related tissue or cells in the individual by any of about 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% or more. The XPO1 inhibitor or an inhibitor of a fragment of XPO1 can be one or more agents selected from the group consisting of a small molecule chemical compound, an antisense oligonucleotide, a siRNA, a non-antibody peptide, or an antibody or functional fragment thereof.

In further embodiments, the neurodegenerative disease is Huntington's Disease and administration of the XPO1 inhibitor or an inhibitor of a fragment of XPO1 to an individual diagnosed with or thought to have Huntington's Disease results in increased clearance of poly-glutamine(Q) protein (Q35 and/or Q40) in the neurons or neural-related tissue or cells in the individual by any of about 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% or more. The XPO1 inhibitor or an inhibitor of a fragment of XPO1 can be one or more agents selected from the group consisting of a small molecule chemical compound, an antisense oligonucleotide, a siRNA, a non-antibody peptide, or an antibody or functional fragment thereof.

### B. Methods for Increasing the Longevity of a Cell

Also provided herein are methods for increasing the longevity of a cell or organism comprising contacting the cell with or administering to the organism an inhibitor of exportin-1 (XPO1). The XPO1 inhibitor or an inhibitor of a fragment of XPO1 can be one or more agents selected from the group consisting of a small molecule chemical compound, an antisense oligonucleotide, a siRNA, a non-antibody peptide, or an antibody or functional fragment thereof.

In some embodiments, the longevity of the cell or organism is increased by any of about 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% or more, relative to cells or organisms that are not contacted with the XPO1 inhibitor or an inhibitor of a fragment of XPO1. Cellular longevity can be assayed by any means known in the art. In some embodiments, the cell is a mammalian cell, an insect cell, a fish cell, or a nematode cell. In other embodiments, the organism is a mouse, rat, or other mammal, a fruitfly (such as D. melanogaster), a zebrafish, or a nematode worm, such as C. elegans. In other embodiments, the organism is a human.

In certain embodiments, the longevity of cells in a tissue may be increased by contacting the tissue with an inhibitor of XPO1 or an inhibitor of a fragment of XPO1. The tissue can be one or more of, without limitation, neural tissue, intestinal tissue, muscle tissue (such as cardiac, smooth, or skeletal muscle tissue), or hypodermal or skin tissue.

In a particular embodiment, the methods provided herein include increasing the longevity of cells in skin tissue by contacting the skin with an inhibitor of XPO1 or an inhibitor of a fragment of XPO1 to prevent, reverse, or reduce a sign of skin aging. As the outermost organ, the skin forms a protective barrier to protect the body from harm. Skin is subject to abuse from both external and internal factors, which can result in skin aging. Skin aging occurs in two ways: (1) chronological aging (i.e., the natural aging process) and (2) through UV rays in sunlight, which accelerate the natural aging process (i.e., photoaging). Chronological aging may result in thinning, loss of elasticity, and/or general degradation of the skin. By contrast, photoaging, which happened in areas of habitual sun exposure, may result in changes such as elastosis, atrophy, wrinkling, vascular changes (i.e., diffuse erythema, ecchymosis, and telangiectasias), pigmentary changes (i.e., lentigines, freckles, and areas of hypo- and hyper-pigmentation), and/or the development of seborrheic keratosis, actinic keratosis, comedones, and cysts.

Consequently, in certain embodiments, the methods provided herein are directed to preventing, alleviating, or reversing one or more signs of skin aging in an individual by contacting the skin of the individual with an inhibitor of XPO1 or an inhibitor of a fragment of XPO1. “Signs of skin aging” as used herein include, but are not limited to, all outward visibly and tactilely perceptible manifestations as well as any other macro or micro effects due to skin aging. Such signs may be induced or caused by intrinsic factors (showing as chronological aged skin) and extrinsic factors (showing as environmental skin damage including but not limited photo-aged skin). These signs may result from processes which include, but are not limited to, the development of textural discontinuities such as wrinkles and coarse deep wrinkles, fine or skin lines, crevices, bumps, large pores (e.g., associated with adnexal strictures such as sweat gland ducts, sebaceous glands, or hair follicles), or unevenness or roughness, loss of skin elasticity (loss and/or inactivation of functional skin elastin), sagging (including puffiness in the eye area and jowls), loss of skin firmness, loss of skin tightness, loss of skin recoil from deformation, discoloration (including under eye circles), blotching, sallowness, hyperpigmented skin regions such as age spots and freckles, keratoses, abnormal differentiation, hyperkeratinization, elastosis, collagen breakdown, and other histological changes in the stratum corneum, dermis, epidermis, the skin vascular system (e.g., telangiectasia or spider vessels), and underlying tissues. especially those proximate to the skin.

### C. Identification of Genes Regulated by HLH30/Transcription Factor EB (TFEB) and/or DAF-16/Forkhead Box Protein O (FOXO)

Previously, it was found that HLH-30/TFEB interacts with DAF-16/FOXO in the nucleus of long-lived animals (Lin X. et al. in preparation) and DAF-16/FOXO is also found in the nucleus of animals. As such, without being bound to theory, it is hypothesized that DAF-16 and HLH-30 co-regulate autophagy targets upon XPO1 silencing. In order to take advantage of the fact that XPO1 inhibition results in the accumulation and increased expression of HLH-30/TFEB in the nuclei of cells, provided herein are methods for identifying one or more genes regulated by HLH30/Transcription factor EB (TFEB) and/or DAF-16/Forkhead box protein O (FOXO), or an orthologue thereof. In some embodiments, the cell is contacted with an inhibitor of XPO1, or an orthologue thereof and one or more genes whose expression changes following contact with the inhibitor of XPO1 is identified using any means known in the art (such as microarray, ChIP-Seq, or quantitative PCR). The XPO1 inhibitor can be one or more agents selected from the group consisting of a small molecule chemical compound, an antisense oligonucleotide, a siRNA, a non-antibody peptide, or an antibody or functional fragment thereof.

In some embodiments, expression of said one or more genes changes due to increased expression or nuclear localization of HLH30/TFEB and/or DAF-16/FOXO following inhibition of XPO1. Any model organism or cell can be used for identifying one or more genes regulated by HLH30/Transcription factor EB (TFEB) and/or DAF-16/Forkhead box protein O (FOXO), or orthologues thereof, such as, for example, a mammalian cell, an insect cell, a fish cell, or a nematode cell.

### III. Compositions

The therapeutic methods disclosed herein encompass inhibiting the expression or activity of an XPO1 gene, protein, or fragment thereof for the treatment of a neurodegenerative disease (such as aging-associated neurodegenerative diseases) in a subject in need thereof using one or more XPO1 inhibitor

### A. XPO1 Inhibitors

In some embodiments, XPO1 inhibitors or an inhibitor of a fragment of XPO1 for use in the methods disclosed herein can be, without limitation, an antibody or functional fragment thereof, an non-antibody binding polypeptide, a small molecule chemical compound, and/or an inhibitory nucleic acid.

### 1. Antibodies

The therapeutic methods disclosed herein can comprise inhibiting the expression or activity of an XPO1 protein, or fragment thereof (such as a functional fragment thereof, for example a domain or a phosphorylation or acetylation site on the XPO1 protein), by administering one or more antibodies that bind to and/or prevent an XPO1 protein or fragment thereof from normally functioning in a cellular or physiological context. “Antibody” as used herein is meant to include intact molecules as well as fragments which retain the ability to bind antigen (e.g., Fab and F(ab′) fragments). These fragments are typically produced by proteolytically cleaving intact antibodies using enzymes such as a papain (to produce Fab fragments) or pepsin (to produce F(ab′)₂ fragments). The term “antibody” also refers to both monoclonal antibodies and polyclonal antibodies. Polyclonal antibodies are derived from the sera of animals immunized with the antigen. Monoclonal antibodies can be prepared using hybridoma technology (Kohler, et al., Nature 256:495 (1975)). In general, this technology involves immunizing an animal, usually a mouse, with the CA125 peptide. The splenocytes of the immunized animals are extracted and fused with suitable myeloma cells, e.g., SP2O cells. After fusion, the resulting hybridoma cells are selectively maintained in a culture medium and then cloned by limiting dilution (Wands, et al., Gastroenterology 80:225-232 (1981)). The cells obtained through such selection are then assayed to identify clones which secrete antibodies capable of binding to XPO1 proteins or fragments thereof.

### 2. Non-Antibody Binding Polypeptides

The therapeutic methods disclosed herein can comprise inhibiting the expression or activity of an XPO1 protein, or fragment thereof (such as a functional fragment thereof, for example a domain or a phosphorylation or acetylation site on the XPO1 protein), by administering one or more non-antibody binding polypeptide antibodies that bind to and/or prevent an XPO1 protein or fragment thereof from normally functioning in a cellular or physiological context. Binding polypeptides are polypeptides that bind, preferably specifically, to an XPO1 protein or fragments thereof. Binding polypeptides may be chemically synthesized using known polypeptide synthesis methodology or may be prepared and purified using recombinant technology. Binding polypeptides are usually at least about 5 amino acids in length, alternatively at least about 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 amino acids in length or more, wherein such binding polypeptides that are capable of binding, preferably specifically, to an XPO1 protein or a fragment thereof. Binding polypeptides may be identified without undue experimentation using well known techniques. In this regard, it is noted that techniques for screening polypeptide libraries for binding polypeptides that are capable of binding to a polypeptide target are well known in the art (see, e.g., U.S. Pat. Nos. 5,556,762, 5,750,373, 4,708,871, 4,833,092, 5,223,409, 5,403,484, 5,571,689, 5,663,143; PCT Publication Nos. WO 84/03506 and WO84/03564; Cwirla, S. E. et al., (1990) Proc. Natl. Acad. Sci. USA, 87:6378; Lowman, H. B. et al., (1991) Biochemistry, 30:10832; Clackson, T. et al., (1991) Nature, 352: 624; Marks, J. D. et al., (1991), J. Mol. Biol., 222:581; Kang, A. S. et al., (1991) Proc. Natl. Acad. Sci. USA, 88:8363, and Smith, G. P. (1991) Current Opin. Biotechnol., 2:668).

### 3. Small Molecule Chemical Compounds

The therapeutic methods disclosed herein can comprise inhibiting the expression or activity of an XPO1 protein, or fragment thereof (such as a functional fragment thereof, for example a domain or a phosphorylation or acetylation site on the XPO1 protein), by administering one or more small molecule chemical compounds that bind to and/or prevent an XPO1 protein or fragment thereof from normally functioning in a cellular or physiological context.

The small molecule chemical compound may be a component of a combinatorial chemical library. Combinatorial chemical libraries are a collection of multiple species of chemical compounds comprised of smaller subunits or monomers. Combinatorial libraries come in a variety of sizes, ranging from a few hundred to many hundreds of thousand different species of chemical compounds. There are also a variety of library types, including oligomeric and polymeric libraries comprised of compounds such as carbohydrates, oligonucleotides, and small organic molecules, etc. Such libraries have a variety of uses, such as immobilization and chromatographic separation of chemical compounds, as well as uses for identifying and characterizing ligands capable of binding an acceptor molecule or mediating a biological activity of interest (such as, but not limited to, the prevention of neurodegeneration that accompanies diseases associated with aging).

Various techniques for synthesizing libraries of compounds on solid-phase supports are known in the art. Solid-phase supports are typically polymeric objects with surfaces that are functionalized to bind with subunits or monomers to form the compounds of the library. Synthesis of one library typically involves a large number of solid-phase supports. To make a combinatorial library, solid-phase supports are reacted with one or more subunits of the compounds and with one or more numbers of reagents in a carefully controlled, predetermined sequence of chemical reactions. In other words, the library subunits are “grown” on the solid-phase supports. The larger the library, the greater the number of reactions required, complicating the task of keeping track of the chemical composition of the multiple species of compounds that make up the library.

Small molecules may be identified and chemically synthesized using known methodology (see, e.g., International Patent Application Publication Nos. WO00/00823 and WO00/39585). A small molecule inhibitor is are less than about 2000 Daltons in size. For example, the inhibitor is less than about 1500, 750, 500, 250 or 200 Daltons in size, wherein such small molecules that are capable of binding, preferably specifically, to an XPO1 gene, protein, or fragment thereof as described herein may be identified without undue experimentation using well known techniques. As used herein, “small molecule chemical compound” excludes proteins.

In this regard, it is noted that techniques for screening small molecule libraries for molecules that are capable of binding to a polypeptide target are well known in the art (see, e.g., PCT Publication Nos. WO2000/00823 and WO2000/39585). Small molecules may be, for example, aldehydes, ketones, oximes, hydrazones, semicarbazones, carbazides, primary amines, secondary amines, tertiary amines, N-substituted hydrazines, hydrazides, alcohols, ethers, thiols, thioethers, disulfides, carboxylic acids, esters, amides, ureas, carbamates, carbonates, ketals, thioketals, acetals, thioacetals, aryl halides, aryl sulfonates, alkyl halides, alkyl sulfonates, aromatic compounds, heterocyclic compounds, anilines, alkenes, alkynes, diols, amino alcohols, oxazolidines, oxazolines, thiazolidines, thiazolines, enamines, sulfonamides, epoxides, aziridines, isocyanates, sulfonyl chlorides, diazo compounds, acid chlorides, or the like.

In some embodiments, the small molecule is a compound of the structural formula I:

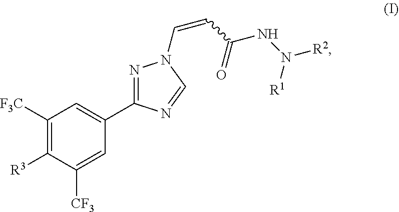

or a pharmaceutically acceptable salt thereof, wherein:
R¹ is selected from hydrogen and methyl;
R² is selected from pyridin-2-yl, pyridin-3-yl, pyridin-4-yl, pyrazin-2-yl, and quinoxalin-2-yl, pyrimidin-4-yl, 1,1-dioxotetrahydrothiophen-3-yl and cyclopropyl, wherein R is optionally substituted with one or more independent substituents selected from methyl and halogen; or
R¹ and R² are taken together with their intervening atoms to form 4-hydroxypiperidin-1-yl, pyrrolidin-1-yl, azepan-1-yl, 4-benzylpiperazin-1-yl, 4-ethylpiperazin-1-yl, 3-hydroxyazetidin-1-yl, or morpholin-4-yl;
R³ is selected from hydrogen and halo; and [symbol]
   represents a single bond wherein a carbon-carbon double bond bound thereto is in an (E)- or (Z)-configuration.

In other embodiments, the small molecule chemical compound is Selinexor (KPT-330):

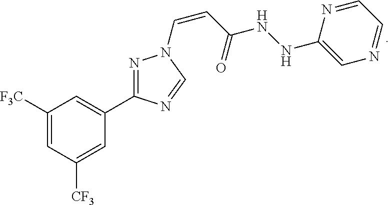 [SMILES: O=C(/C=C\n1cnc(-c2cc(C(F)(F)F)cc(C(F)(F)F)c2)n1)NNc1cnccn1]

In further embodiments, the small molecule chemical compound is Verdinexor (KPT-335):

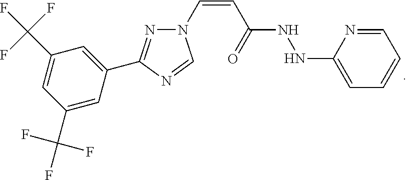 [SMILES: O=C(/C=C\n1cnc(-c2cc(C(F)(F)F)cc(C(F)(F)F)c2)n1)NNc1ccccn1]

In some embodiments, the small molecule is a compound of the structural formula II:

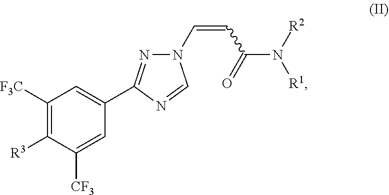

or a pharmaceutically acceptable salt thereof, wherein:
R¹ is selected from hydrogen and methyl;
R² is selected from pyridin-2-yl, pyridin-3-yl, pyridin-4-yl, pyrazin-2-yl, and quinoxalin-2-yl, pyrimidin-4-yl, 1,1-dioxotetrahydrothiophen-3-yl and cyclopropyl, wherein R is optionally substituted with one or more independent substituents selected from methyl and halogen; or
R¹ and R² are taken together with their intervening atoms to form 4-hydroxypiperidin-1-yl, pyrrolidin-1-yl, azepan-1-yl, 4-benzylpiperazin-1-yl, 4-ethylpiperazin-1-yl, 3-hydroxyazetidin-1-yl, azetidin-1-yl, or morpholin-4-yl, optionally substituted with 1, 2, 3, or 4 fluorines;
R³ is selected from hydrogen and halo; and [symbol]
   represents a single bond wherein a carbon-carbon double bond bound thereto is in an (E)- or (Z)-configuration.

In some embodiments, the small molecule chemical compound is KPT-276:

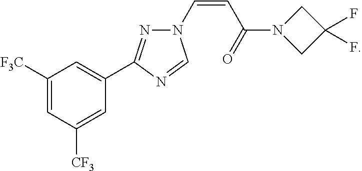 [SMILES: O=C(/C=C\n1cnc(-c2cc(C(F)(F)F)cc(C(F)(F)F)c2)n1)N1CC(F)(F)C1]

In some embodiments, the small molecule is a compound of the structural formula III:

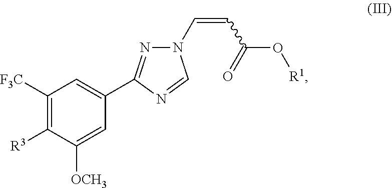

or a pharmaceutically acceptable salt thereof, wherein:
R¹ is an alkyl group having from 1 to 6 carbons;
R³ is selected from hydrogen and halo; and [symbol]
   represents a single bond wherein a carbon-carbon double bond bound thereto is in an (E)- or (Z)-configuration.

In some embodiments, the small molecule chemical compound is KPT-185:

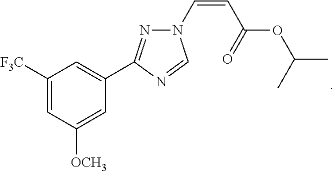 [SMILES: COc1cc(-c2ncn(/C=C\C(=O)OC(C)C)n2)cc(C(F)(F)F)c1]

In some embodiments, the small molecule chemical compound inhibitor of XPO1 (such as, Selinexor) is orally bioavailable. In some embodiments, the small molecule chemical compound is lipophilic or is formulated with one or more lipophilic excipients or vehicles, such as (without limitation), glycerol stearates, palmitostearates and behenates; hydrogenated vegetable oils and their derivatives; vegetable and animal wax and their derivatives; hydrogenated castor oils and their derivatives and cetylic esters and/or alcohols) to render a formulation comprising the small molecule chemical compound inhibitor of XPO1 lipophilic to cross the blood brain barrier.

### 4. Inhibitory Nucleic Acids

The therapeutic methods disclosed herein can comprise inhibiting the expression or activity of an XPO1 protein, or fragment thereof, by administering one or more inhibitory nucleic acids directed to an XPO1 DNA or RNA. Such nucleic acids can include, without limitations, antisense oligonucleotides, small inhibitory RNAs (siRNAs), triplex-forming oligonucleotides, ribozymes, or any other inhibitory oligonucleotide or nucleic acid. In addition, the nucleic acid-based therapeutics for use in the methods described herein can have one or more alterations to the oligonucleotide phosphate backbone, sugar moieties, and/or nucleobase (such as any of those described herein) that increase resistance to degradation, such as by nuclease cleavage. Nucleic acids complementary to the XPO1 gene or RNA are at least about 10 (such as any of about 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50) nucleotides in length. In another embodiment, the nucleic acids can be between about 10-15, 15-20, 20-25, 25-30, 30-35, 35-40, 40-45, or 45-50 oligonucleotides in length.

The naturally occurring internucleoside linkage of RNA and DNA is a 3′ to 5 phosphodiester linkage. The nucleic acids used according to any of the methods disclosed herein can have one or more modified, i.e. non-naturally occurring, internucleoside linkages. With respect to therapeutics, modified internucleoside linkages are often selected over oligonucleotides having naturally occurring internucleoside linkages because of desirable properties such as, for example, enhanced cellular uptake, enhanced affinity for target nucleic acids, and increased stability in the presence of nucleases.

Oligonucleotides (such as an antisense oligonucleotide) having modified internucleoside linkages include internucleoside linkages that retain a phosphorus atom as well as internucleoside linkages that do not have a phosphorus atom. Representative phosphorus containing internucleoside linkages include, but are not limited to, phosphodiesters, phosphotriesters, methylphosphonates, phosphoramidate, and phosphorothioates. Methods of preparation of phosphorous-containing and non-phosphorous-containing linkages are well known.

As is known in the art, a nucleoside is a base-sugar combination. The base portion of the nucleoside is normally a heterocyclic base. The two most common classes of such heterocyclic bases are the purines and the pyrimidines. Nucleotides are nucleosides that further include a phosphate group covalently linked to the sugar portion of the nucleoside. For those nucleosides that include a pentofuranosyl sugar, the phosphate group can be linked to either the 2′, 3′ or 5′ hydroxyl moiety of the sugar. In forming oligonucleotides, the phosphate groups covalently link adjacent nucleosides to one another to form a linear polymeric compound. In turn the respective ends of this linear polymeric structure can be further joined to form a circular structure, however, open linear structures are generally preferred. Within the oligonucleotide structure, the phosphate groups are commonly referred to as forming the internucleoside backbone of the oligonucleotide. The normal linkage or backbone of RNA and DNA is a 3′ to 5′ phosphodiester linkage.

Specific though nonlimiting examples of nucleic acids (such as antisense or siRNA oligonucleotides) useful in the methods of the present invention include oligonucleotides containing modified backbones or non-natural internucleoside linkages. As defined in this specification, oligonucleotides having modified backbones include those that retain a phosphorus atom in the backbone and those that do not have a phosphorus atom in the backbone. For the purposes of this specification, and as sometimes referenced in the art, modified oligonucleotides that do not have a phosphorus atom in their internucleoside backbone can also be considered to be oligonucleosides.

In some embodiments, modified oligonucleotide backbones include, for example, phosphorothioates, chiral phosphorothioates, phosphorodithioates, phosphotriesters, aminoalkylphosphotri-esters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, thiono-phosphoramidates, thionoalkylphosphonates, thionoalkylphospho-triesters, selenophosphates and boranophosphates having normal 3′-5′ linkages, 2′-5′ linked analogs of these, and those having inverted polarity wherein one or more internucleotide linkages is a 3′ to 3′, 5′ to 5′ or 2′ to 2′ linkage. Oligonucleotides having inverted polarity comprise a single 3′ to 3′ linkage at the 3′-most internucleotide linkage i.e. a single inverted nucleoside residue which may be abasic (the nucleobase is missing or has a hydroxyl group in place thereof) can also be employed. Various salts, mixed salts and free acid forms are also included.

Oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and C component parts.

Representative United States patents that teach the preparation of the above phosphorus-containing and non-phosphorus-containing linkages include, but are not limited to, U.S. Pat. Nos. 5,541,306; 5,550,111; 5,563,253; 5,571,799; 5,587,361; 5,194,599; 5,565,555; 5,527,899; 5,721,218; 5,672,697 and 5,625,050, 5,596,086; 5,602,240; 5,610,289; 5,602,240; 5,608,046; 5,610,289; 5,618,704; 5,623,070; 5,663,312; 5,633,360; 5,677,437; 5,792,608; 5,646,269 and 5,677,439, each of which is herein incorporated by reference.

Modified nucleic acids (such as siRNA or antisense oligonucleotides) complementary to an exportin-1 DNA or RNA sequence used as anticancer therapies in conjunction with any of the methods disclosed herein may also contain one or more substituted or modified sugar moieties. For example, the furanosyl sugar ring can be modified in a number of ways including substitution with a substituent group, bridging to form a bicyclic nucleic acid “BNA” and substitution of the 4′-0 with a heteroatom such as S or N(R) as described in U.S. Pat. No. 7,399,845, hereby incorporated by reference herein in its entirety. Other examples of BNAs are described in published International Patent Application No. WO 2007/146511, hereby incorporated by reference herein in its entirety.

Nucleic acids (such as antisense oligonucleotides) for use in any of the methods disclosed herein may also include nucleobase (often referred to in the art simply as “base”) modifications or substitutions. Nucleobase modifications or substitutions are structurally distinguishable from, yet functionally interchangeable with, naturally occurring or synthetic unmodified nucleobases. Both natural and modified nucleobases are capable of participating in hydrogen bonding. Such nucleobase modifications may impart nuclease stability, binding affinity or some other beneficial biological property to oligonucleotide compounds. Modified nucleobases include synthetic and natural nucleobases such as, for example, 5-methylcytosine (5-me-C). Certain nucleobase substitutions, including 5-methylcytosine substitutions, are particularly useful for increasing the binding affinity of an oligonucleotide compound (such as an antisense oligonucleotide compound or an siRNA) for a target nucleic acid (such as an XPO1 nucleic acid).

Additional unmodified nucleobases include 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C≡C—CH3) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine.

Heterocyclic base moieties may also include those in which the purine or pyrimidine base is replaced with other heterocycles, for example 7-deaza-adenine, 7-deazaguanosine, 2-aminopyridine and 2-pyridone. Nucleobases that are particularly useful for increasing the binding affinity of antisense compounds include 5-substituted pyrimidines, 6-azapyrimidines and N-2, N-6 and 0-6 substituted purines, including 2 aminopropyladenine, 5-propynyluracil and 5-propynylcytosine.

As used herein, “unmodified” or “natural” nucleobases include the purine bases adenine (A) and guanine (G), and the pyrimidine bases thymine (T), cytosine (C) and uracil (U).

In some embodiments, the nucleic acid inhibitor of XPO1 (such as, an siRNA) is orally bioavailable. In some embodiments, the nucleic acid is lipophilic or is formulated with one or more lipophilic excipients or vehicles, such as (without limitation), glycerol stearates, palmitostearates and behenates; hydrogenated vegetable oils and their derivatives; vegetable and animal wax and their derivatives; hydrogenated castor oils and their derivatives and cetylic esters and/or alcohols) to render a formulation comprising the nucleic acid inhibitor of XPO1 lipophilic to cross the blood brain barrier.

### B. Pharmaceutical Compositions

Any of the inhibitors of XPO1 (such as oligonucleotide-based therapies or small molecule chemical compounds, for example, Selinexor) disclosed herein can be administered in the form of pharmaceutical compositions. These compounds can be administered by a variety of routes including oral, rectal, transdermal, subcutaneous, intravenous, intramuscular, and intranasal. These compounds are effective as both injectable and oral compositions. Such compositions are prepared in a manner well known in the pharmaceutical art and comprise at least one active compound. When employed as oral compositions, the oligonucleotides and another disclosed herein are protected from acid digestion in the stomach by a pharmaceutically acceptable protectant.

This invention also includes pharmaceutical compositions which contain, as the active ingredient, one or more of the therapies (such as an inhibitor of XPO1) for treating any of the neurodegenerative disorders or disorders associated with aging disclosed herein along with one or more pharmaceutically acceptable excipients or carriers. In making the compositions of this invention, the active ingredient is usually mixed with an excipient or carrier, diluted by an excipient or carrier or enclosed within such an excipient or carrier which can be in the form of a capsule, sachet, paper or other container. When the excipient or carrier serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders.

In preparing a formulation, it may be necessary to mill the active lyophilized compound to provide the appropriate particle size prior to combining with the other ingredients. If the active compound is substantially insoluble, it ordinarily is milled to a particle size of less than 200 mesh. If the active compound is substantially water soluble, the particle size is normally adjusted by milling to provide a substantially uniform distribution in the formulation, e.g. about 40 mesh.

Some examples of suitable excipients or carriers include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents. The compositions of the invention can be formulated so as to provide quick, sustained or delayed release of the active ingredient after administration to the subject by employing procedures known in the art.

The compositions can be formulated in a unit dosage form, each dosage containing from about 5 mg to about 100 mg or more, such as any of about 1 mg to about 5 mg, 1 mg to about 10 mg, about 1 mg to about 20 mg, about 1 mg to about 30 mg, about 1 mg to about 40 mg, about 1 mg to about 50 mg, about 1 mg to about 60 mg, about 1 mg to about 70 mg, about 1 mg to about 80 mg, or about 1 mg to about 90 mg, inclusive, including any range in between these values, of the active ingredient. The term “unit dosage forms” refers to physically discrete units suitable as unitary dosages for individuals, each unit containing a predetermined quantity of active material calculated to produce the desired therapeutic effect, in association with a suitable pharmaceutical excipient or carrier.

The neurodegenerative disorder (such as a neurodegenerative disorder associated with aging or other aging-associated disorder or disease) therapies disclosed herein are effective over a wide dosage range and are generally administered in a therapeutically effective amount. It will be understood, however, that the amount of the therapies actually administered will be determined by a physician, in the light of the relevant circumstances, including the condition to be treated, the chosen route of administration, the actual compound administered, the age, weight, and response of the individual subject, the severity of the subject's symptoms, and the like.

In some embodiments, the inhibitors of XPO1 disclosed herein (such as oligonucleotide-based therapies or small molecule chemical compounds, for example, Selinexor) may be used in combination with one or more second active agents to treat, prevent, and/or manage neurodegenerative disorders associated with aging or other aging-associated disorder or disease described herein (e.g., AD, ALS, or HD. In certain embodiments, the second active agent is an antipsychotic agent. In certain embodiments, the second active agent is an atypical antipsychotic agent. In certain embodiments, the second active agent is an agent that is useful for the treatment of Alzheimer's disease, Huntington's disease, ALS, or dementia. The second active agent can be formulated to include a cholinesterase inhibitor, an antidepressant agent, an SSRI, SNRI, or tricyclic antidepressant. In certain embodiments, the second active agent is lurasidone, olanzapine, risperidone, aripiprazole, amisulpride, asenapine, blonanserin, clozapine, clotiapine, illoperidone, mosapratnine, paliperidone, quetiapine, remoxipride, sertindole, sulpiride, ziprasidone, zotepine, pimavanserin, loxapine, donepezil, rivastigmine, memantine, galantamine, tacrine, amphetamine, methylphenidate, atomoxetine, modafinil, sertraline, fluoxetine, venlafaxine, duloxetine, or L-DOPA.

The tablets or pills can be coated or otherwise compounded to provide a dosage form affording the advantage of prolonged action and to protect the therapies (such as an oligonucleotide or small molecule chemical compound) from acid hydrolysis in the stomach. For example, the tablet or pill can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

The liquid forms in which the novel compositions of the present invention can be incorporated for administration orally or by injection include aqueous solutions, suitably flavored syrups, aqueous or oil suspensions, and flavored emulsions with edible oils such as corn oil, cottonseed oil, sesame oil, coconut oil, or peanut oil, as well as elixirs and similar pharmaceutical vehicles.

Compositions for inhalation or insufflation include solutions and suspensions in pharmaceutically acceptable, aqueous or organic solvents, or mixtures thereof, and powders. The liquid or solid compositions can contain suitable pharmaceutically acceptable excipients as described herein. The compositions can be administered by the oral or nasal respiratory route for local or systemic effect. Compositions in pharmaceutically acceptable solvents can be nebulized by use of inert gases. Nebulized solutions can be inhaled directly from the nebulizing device or the nebulizing device can be attached to a face mask tent, or intermittent positive pressure breathing machine. Solution, suspension, or powder compositions can also be administered, orally or nasally, from devices which deliver the formulation in an appropriate manner.

In some embodiments, the inhibitor of XPO1 (for example, Selinexor) is orally bioavailable or is formulated for oral bioavailability. In some embodiments, the inhibitor of XPO1 is lipophilic or is formulated with one or more lipophilic excipients or vehicles, such as (without limitation), glycerol stearates, palmitostearates and behenates; hydrogenated vegetable oils and their derivatives; vegetable and animal wax and their derivatives; hydrogenated castor oils and their derivatives and cetylic esters and/or alcohols) to render a formulation comprising the inhibitor of XPO1 lipophilic to cross the blood brain barrier.

It is intended that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

The invention can be further understood by reference to the following examples, which are provided by way of illustration and are not meant to be limiting.

### EXAMPLES

The practice of the present invention will employ, unless otherwise indicated, conventional techniques of molecular biology, microbiology, cell biology, biochemistry, nucleic acid chemistry, and immunology, which are well known to those skilled in the art. Such techniques are explained fully in the literature, such as, Molecular Cloning: A Laboratory Manual, fourth edition (Sambrook et al., 2012) and Molecular Cloning: A Laboratory Manual, third edition (Sambrook and Russel, 2001), (jointly referred to herein as “Sambrook”); Current Protocols in Molecular Biology (F. M. Ausubel et al., eds., 1987, including supplements through 2014); PCR: The Polymerase Chain Reaction, (Mullis et al., eds., 1994); Antibodies: A Laboratory Manual, Second edition; Cold Spring Harbor Laboratory Press, Cold Spring Harbor, N.Y. (Greenfield, ed., 2014), Beaucage et al. eds., Current Protocols in Nucleic Acid Chemistry, John Wiley & Sons, Inc., New York, 2000, (including supplements through 2014) and Gene Transfer and Expression in Mammalian Cells (Makrides, ed., Elsevier Sciences B.V., Amsterdam, 2003).

### Example 1

This example illustrates the identification of xpo-1, an ortholog of mammalian exportin-1 (XPO1), as a potent inducer of the nuclear localization of HLH-30. The transcription factor HLH-30/TFEB modulates autophagy and lysosomal gene expression (FIG. 1A) and provides an attractive target for the modulation of the autophagy process. HLH-30/TFEB is enriched in the nucleus in autophagy-stimulated conditions (starvation) and in long-lived animals.

### Materials and Methods

The nematode C. elegans is an art-recognized system to identify genetic and pharmacological modifiers of various cellular processes. As such, an unbiased genome-wide RNAi screen for genetic modifiers of the activity of HLH-30/TFEB was performed (FIG. 1B). Specifically, enhancers of HLH-30 nuclear localization were searched for by following the distribution of HLH-30 fused to GFP.

Nematode maintenance: Strains of C. elegans were maintained at 20° C. on agar plates seeded with OP50 E. coli and handled as originally described (Brenner, S. (1974). The genetics of Caenorhabditis elegans. Genetics 77, 71-94.). See Table 1 for strains used throughout Examples section.

TABLE 1
C. elegans strains used throughout Examples.
Strains used in this study

| | | |
|---|---|---|
| N2 | Wild-type (Kenyon lab - CF) | — |
| AA292 | daf-36(k114) V | — |
| AM140 | rmIs132 [unc-54p::Q35::YFP] | — |
| CF1037 | daf-16(mu86) I | — |
| CF1903 | glp-1(e2144ts) III* | — |
| CF1908 | eat-2(ad1116) II | — |
| CF1934 | daf-16(mu86) I; muIs109[Pdaf-16::gfp::daf-16cDNA + Podr-1::rfp] | |
| GF78 | dgEx78 [(pAMS68) vha-6p::Q40::YFP + rol-6(su1006)] | — |
| GMC101 | dvIs100 [unc-54p::A-beta-1-42::unc-54 3′-UTR + mtl-2p::GFP] | |
| LRL1 | hlh-30(tm1978) IV | — |
| LRL9 | atg-7(bp411) IV | — |
| LRL12 | hlh-30(tm1978) IV; sqIs11[lgg-1p::mcherry::gfp::lgg-1 + rol-6(su1006)] | |
| MAH215 | sqIs11[lgg-1p::mcherry::gfp::lgg-1 + rol-6(su1006)] | — |
| MAH240 | sqls17[hlh-30p::hlh-30::GFP + rol-6(su1006)] | — |
| VB633 | rsks-1(sv31) III | — |
| VC893 | atg-18(gk378) V | — |

*CF1903 was originally classified as glp-1(e2141ts). Upon sequencing, it was found to instead carry the e2144ts allele (see Caenorhabditis Genetics Center website).

HLH-30-GFP-expressing nematodes were fed HT115 E. coli bacteria transformed with a plasmid containing the L4440 backbone and a portion of the cDNA sequence complementary to the coding sequence of the xpo-1 gene. Nuclear enrichment was measured by fluorescent microscopy after animals were treated from eggs to day 1 of adulthood. The RNAi was based on the cosmid sequence ZK742.1 of the C. elegans genome. The sequence of the RNAi used is:

(SEQ ID NO: 5)
CGCGCGTATACGACTCNCTATAGGGCGAATTGGGTACCGGGCCCCCCCTC
GAGGTCGACGGTATCGA
TAAGCTTGATTCTCGTGCATGAACTCGAAAAGCTTATTGATAACCGTCTT
CAGGAACTTCCAGTGAGCTCGAAGGAATCTCGGGTACTGTCCGACAACAT
ACATGATGTTTGACGCAATCACTGCCTTATTGTCCTTTCCACGTTTCTGT
TCACAGAGCCCGAGTAAATCACGAATAACAAGAACAAGGAATCGCTTTTC
GTCTTCTTCAACCATCGTTCCAGAAATCGAACCAACAGCCCAGCACAAAC
GATTCAAATTCTTCCACGAAAACTCTCCTCCGTTCACTTGTGATGCCAAC
TTTTCAGTCATCTTCACTTCAGTGTCCTTGTTGTCAAGATGAGTCAAATA
GACAAGCGTTTCACGCATGTTACGGTACAAAGCAATCGAATCAGTATCTT
TGACCATTTCACGAACAACTTCTCCTTGATCATTTTCCACAATCAACACC
TCTTCTGGCTTTGCCATTCGAGAAATCATT
GTTGAACGAAGTTGCGAGAGGTATTCACGGTAGAGTTGACGACGTGGATG
CTCACGAACCTGACTCATCATTCCGTAAAGAGTACTTGGCTGAATGAATG
GACATATACGGTAGAGCTCAGCAGTCAACCAGCACCAACAATCGAGGCAA
ACTTTGAAAACCTCCATTTCTTCGATCAAAGTGATTTTCAAAAGAAGTTG
AATAGCATAGTCGTGAGATTCTCGCATTAAAATTTTAGCCTCAGTTAATG
GCTCATCAGTTACTTCAATCAGATGAACGTGCTCTTTGATGAACGCGACA
AGAAATTGAGCCAGACTACTGATGAGTTTCTGATCCTGATCGGAAGCATC
TTTGTAGACAGCAGCCAGGTCGAGGTCCAAAGATAGAACTTGGCTAATGT
GGCGCATTGTAGAGC

Fluorescent microscopy imaging: Transgenic worms were mounted on a 2% agarose pad with 0.1% sodium azide and imaged using a Zeiss Discovery V20 fluorescent microscope.

Gene expression analyses: Worms were collected, washed in M9 buffer and worm pellets were flash frozen with liquid nitrogen. RNA was extracted as described previously (Lapierre et al. (2013). The TFEB orthologue HLH-30 regulates autophagy and modulates longevity in Caenorhabditis elegans. Nat Commun 4, 2267.). See Table 2 for details.

TABLE 2
cDNA were prepared using the iScript Reverse Transcriptase Kit (Bio-
Rad). Diluted cDNA of biological quadruplicates were prepared (1/100
dilution) and loaded onto a 96-well plate as technical duplicates.
Serial diluted standards (1/25 to 1/400 of pooled cDNA) were included
in each 96-well cDNA plates and used to calculate primer efficiency
and determine relative levels of mRNA. Diluted samples were loaded on
qPCR plates using a Hydra Matrix (Thermo Fisher Scientific).
SsoAdvanced Universal SYBR Green Supermix (Bio-Rad) and corresponding
primers (above) were added and qPCR plates were run using a Roche 96
Lightcycler. Results from genes of interest were normalized using the
geometric mean of 4 housekeeping genes (act-1, cyn-1, cdc-42, pmp-3).

| Gene | Forward Primer | Reverse Primer | Temp. ° C. |
|---|---|---|---|
| act-1 | CTACGAACTTCCTGACGGACAAG | CCGGCGGACTCCATACC | 60 |
| | (SEQ ID NO: 6) | (SEQ ID NO: 7) | |
| cyn-1 | GTGTCACCATGGAGTTGTTC | TCCGTAGATTGATTCACCAC | 60 |
| | (SEQ ID NO: 8) | (SEQ ID NO: 9) | |
| cdc-42 | CTGCTGGACAGGAAGATTACG | CTCGGACATTCTCGAATGAAG | 60 |
| | (SEQ ID NO: 10) | (SEQ ID NO: 11) | |
| PmP-3 | GTTCCCGTGTTCATCACTCAT | ACACCGTCGAGAAGCTGTAGA | 60 |
| | (SEQ ID NO: 12) | (SEQ ID NO: 13) | |
| xpo-1 | AAGAACAGGCCGAGGCTAAC | GTTGGACTTGTGGCAACGAC | 60 |
| | (SEQ ID NO: 14) | (SEQ ID NO: 15) | |
| Igg-1 | ACCCAGACCGTATTCCAGTG | ACGAAGTTGGATGCGTTTTC | 60 |
| | (SEQ ID NO: 16) | (SEQ ID NO: 17) | |
| Igg-2 | GCATATAACCGTTGCCGAGC | CAAAGCCATCTGGATCACGC | 60 |
| | (SEQ ID NO: 18) | (SEQ ID NO: 19) | |
| sqst-1 | TGGCTGCTGCATCATCCGCT | TCAATCGTGCCGAGACCGGG | 60 |
| | (SEQ ID NO: 20) | (SEQ ID NO: 21) | |
| hlh-30 | CTCATCGGCCGGCGCTCATC | AGAACGCGATGCGTGGTGGG | 60 |
| | (SEQ ID NO: 22) | (SEQ ID NO: 23) | |
| lipl-1 | TGCAACACGGTCTTGAATGC | CCAATCCCAGAATGCCGAGT | 60 |
| | (SEQ ID NO: 24) | (SEQ ID NO: 25) | |
| lipl-2 | GTTGCTAGCATGTGCCAGTG | TGCCAGAAAGCAGTTTCCGA | 60 |
| | (SEQ ID NO: 26) | (SEQ ID NO: 27) | |
| lipl-3 | CGATGGGGTTATCCGGCAAT | CGGGCAGGTTCATAGTCCAG | 60 |
| | (SEQ ID NO: 28) | (SEQ ID NO: 29) | |
| lipl-4 | ACAGGTATTGCGGATGTTTCC | GCATTTGTTCCCCAAATGAA | 60 |
| | (SEQ ID NO: 30) | (SEQ ID NO: 31) | |

Autophagy analysis: Worms expressing the tandem reporter GFP-mCherry-LGG-1 (Chang et al., (2017). Spatiotemporal regulation of autophagy during Caenorhabditis elegans aging. eLife 6) were treated from L4/day 1 of adulthood and imaged using an LSM 800 Zeiss Confocal Laser Scanning Microscope as previously described (Chang et al., (2017). Spatiotemporal regulation of autophagy during Caenorhabditis elegans aging. eLife 6).

Proteostasis analyses: Heat shock analyses were performed at 37° C. and scoring survival every hour. Aggregation was visualized by fluorescent microscopy using strains expressing Q35::GFP in muscle (AM140) (Morley et al., 2002, The threshold for polyglutamine-expansion protein aggregation and cellular toxicity is dynamic and influenced by aging in Caenorhabditis elegans. Proc Natl Acad Sci USA 99, 10417-10422) or Q40::YFP in intestine (GF78) (Mohri-Shiomi and Garsin, (2008) Insulin signaling and the heat shock response modulate protein homeostasis in the Caenorhabditis elegans intestine during infection. J Biol Chem 283, 194-201). Aggregate clearance was assayed with a strain (GMC101) (McColl et al., (2012). Utility of an improved model of amyloid-beta (Abeta(1)(−) (4)(2)) toxicity in Caenorhabditis elegans for drug screening for Alzheimer's disease. Mol Neurodegener 7, 57). Utility of an improved model of amyloid-beta (Abeta(1)(−)(4)(2)) toxicity in Caenorhabditis elegans for drug screening for Alzheimer's disease. Mol Neurodegener 7, 57) inducing expression of human Aβ42 at 25° C. Following a 5-day RNAi feeding, paralysis was assayed after 48 hours at 25° C.

Lifespan analysis: Worms were synchronized by bleaching and eggs were grown on OP50 E. coli during development. Day 1 adults were transferred onto corresponding RNAi plates and kept at 20° C. Mantel-Cox log rank statistical analyses were calculated using Stata 13.0.

### Results

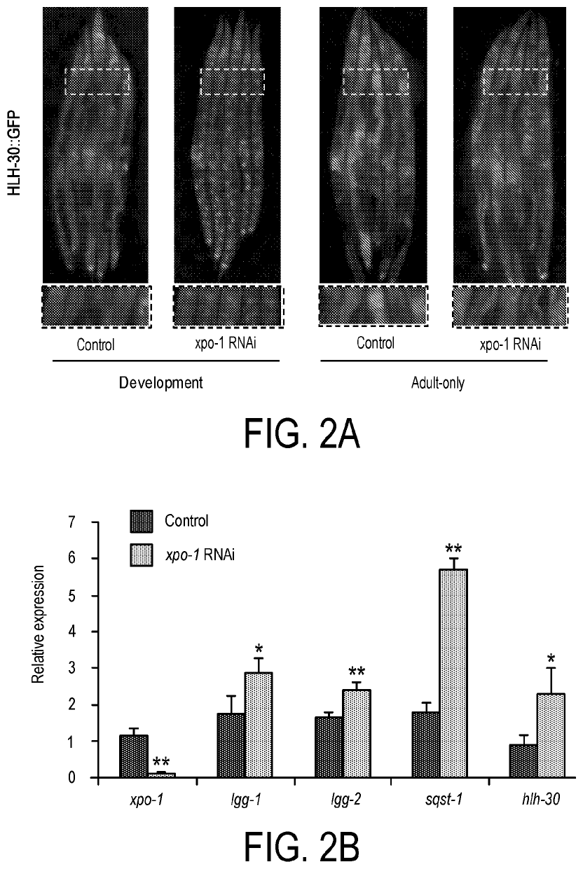

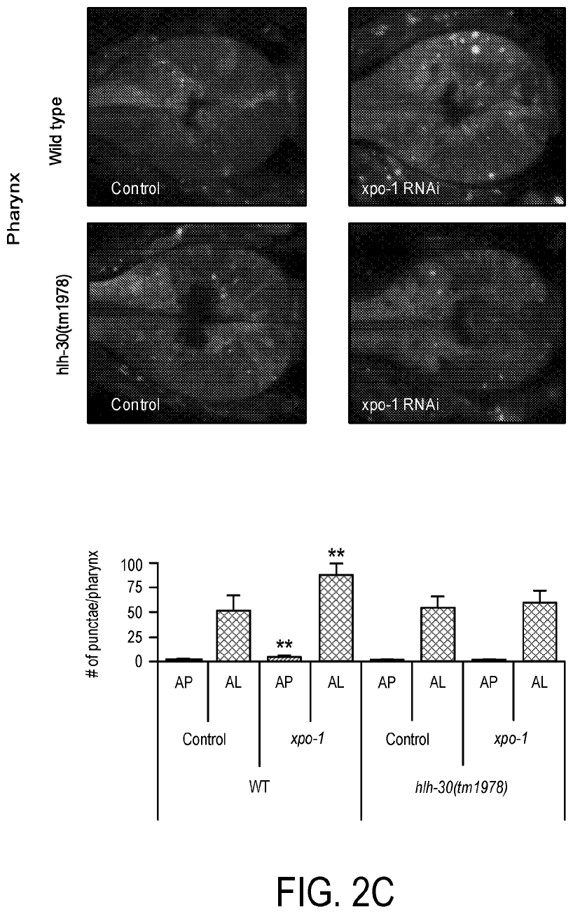

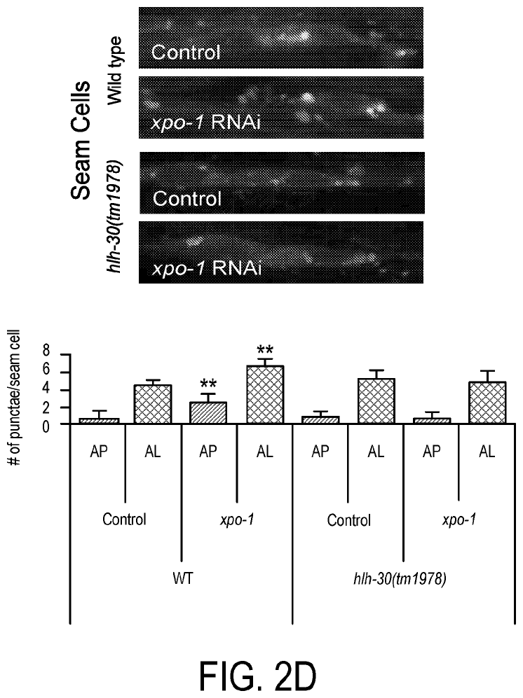

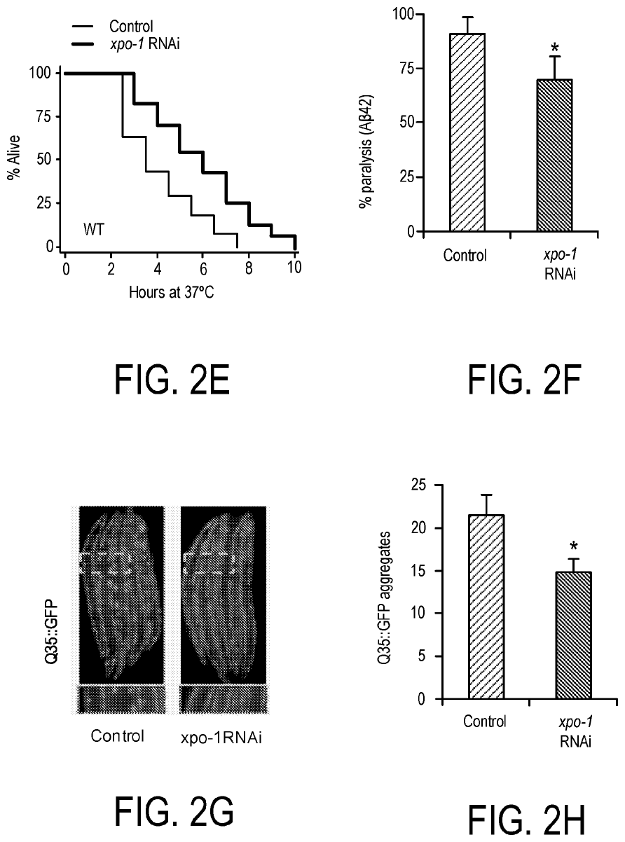

Among several modulators uncovered, silencing of xpo-1, an ortholog of mammalian exportin-1, was the most potent inducer of the nuclear localization of HLH-30 (FIG. 2A). Enrichment of the reporter HLH-30::GFP in intestinal nuclei was found in 100% of treated animals, as previously observed in multiple longevity models (Lapierre, L. R. et al. The TFEB orthologue HLH-30 regulates autophagy and modulates longevity in Caenorhabditis elegans. Nat Commun 4, 2267 (2013)).

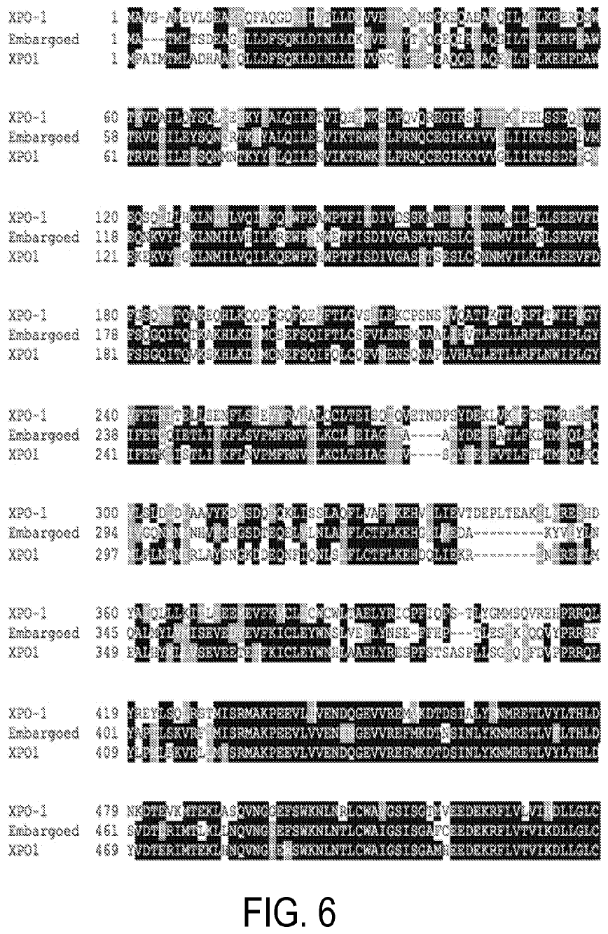

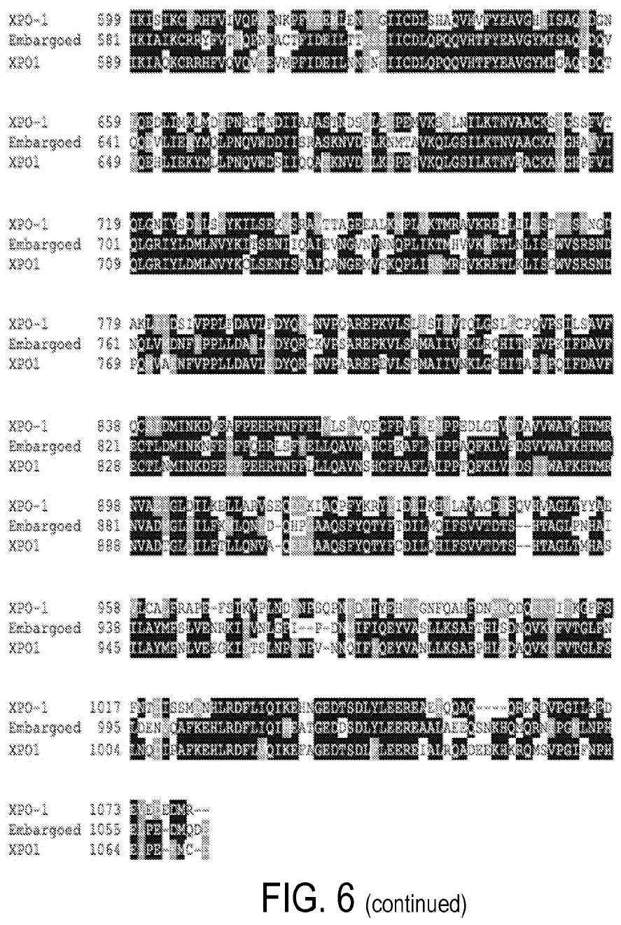

Moreover, as measured by qPCR, silencing of xpo-1, a conserved ortholog of mammalian Exportin-1 (XPO1/CRM-1) (FIG. 6), led to the induction of gene expression for several proteins known to be involved in autophagy, such as HLH-30, P62, and SUL-2 (FIG. 2B). As such, this Example demonstrates that silencing xpo-1 leads to 1) nuclear enrichment of HLH-30—(the ortholog of mammalian TFEB) and induction of the expression of autophagy genes, such as sqst-1 (the orthologue of mammalian P62), and sul-2 (the orthologue of mammalian ARSA).

Xpo-1 was the most potent inducer of the nuclear localization of HLH-30 (FIG. 2A) as enrichment was found in 100% of treated animals, exceeding reported levels in longevity models (Lapierre et al., (2013). The TFEB orthologue HLH-30 regulates autophagy and modulates longevity in Caenorhabditis elegans. Nat Commun 4, 2267; Nakamura et al., (2016). Mondo complexes regulate TFEB via TOR inhibition to promote longevity in response to gonadal signals. Nat Commun 7, 10944). Expression of key autophagy genes and HLH-30/TFEB targets, such as lgg-1 and lgg-2 (LC3/GABARAP), sqst-1(SQSTM-1/p62) and hlh-30(TFEB) were significantly increased upon xpo-1 silencing (FIG. 2B). To measure autophagy directly, a tandem autophagy reporter (GFP::mCherry::LGG-1) was used that reports autophagosome and autolysosome formation (Chang et al., (2017). Spatiotemporal regulation of autophagy during Caenorhabditis elegans aging. eLife 6). Autophagosome (AP) and autolysosome formation (AL) were enhanced in the pharynx and hypodermal seam cells of wild-type animals treated with xpo-1 RNAi (FIG. 2C and FIG. 2D). Silencing xpo-1 in hlh-30(tm1978) mutants failed to enhance autophagy (FIG. 2C and FIG. 2D), demonstrating a direct role for HLH-30 activity in autophagic induction.

Animals subjected to xpo-1 RNAi displayed increased heat resistance, consistent with a role for enhanced autophagy in heat resistance (Kumsta et al., (2017). Hormetic heat stress and HSF-1 induce autophagy to improve survival and proteostasis in C. elegans. Nat Commun 8, 14337; Visvikis et al., (2014). Innate Host Defense Requires TFEB-Mediated Transcription of Cytoprotective and Antimicrobial Genes. Immunity. 40 896-909) (FIG. 2E). Silencing xpo-1 decreased paralysis from expressing Alzheimer's disease-related protein Aβ342 (McColl et al., (2012). Utility of an improved model of amyloid-beta (Abeta(1)(−)(4)(2)) toxicity in Caenorhabditis elegans for drug screening for Alzheimer's disease. Mol Neurodegener 7, 57) (FIG. 2F) and lowered the formation of Huntington's disease-like polyQ-containing protein aggregates (Q35::GFP) (Morley et al., (2002). The threshold for polyglutamine-expansion protein aggregation and cellular toxicity is dynamic and influenced by aging in Caenorhabditis elegans. Proc Natl Acad Sci USA 99, 10417-10422) (FIG. 2G and FIG. 2H). Altogether, this data establish a novel role for the nuclear export protein XPO-1/XPO1 in the modulation of autophagy and proteostasis by regulating the nuclear localization and the activity of HLH-30/TFEB.

### Example 2

This Example shows the functional assessment of the effects of genetically or pharmacologically inhibiting xpo-1 on the autophagic pathway and for longevity in C. elegans.

### Materials and Methods

To functionally assess the effect of genetically or pharmacologically inhibiting xpo-1 on the autophagic pathway, autophagic flux (i.e. fusion of autophagosome to lysosome) was measured in C. elegans. Several fluorescent reporters of autophagy proteins (including LGG-1/LC3, SQST-1/P62) have been designed to observe and quantify autophagic flux.

A tandem reporter GFP-mCherry-LGG-1 that displays and distinguishes autophagosome (green and red) and autolysosomes (red) was expressed in nematodes. These animals were used to determine the effect of silencing (RNAi) xpo-1 on autophagy using fluorescent microscopy. The accumulation of unique red punctae represents active autolysosome formation, indicative of activated autophagy.

### Results

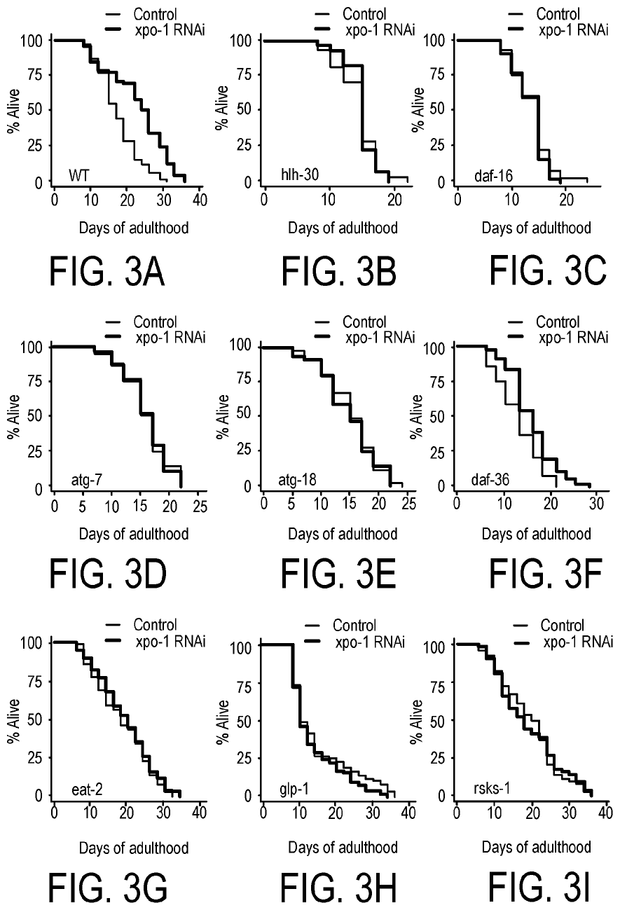

As shown in FIG. 2C and FIG. 2D, silencing of xpo-1 by siRNA enhances autophagy. Further, as shown in FIG. 3A, xpo-1 silencing leads to lifespan extension in C. elegans.

### Example 3

To better understand the cytoprotective effects associated with xpo-1 silencing, the effects of xpo-1 inhibition on the clearance of intrinsically disordered proteins expressed in a C. elegans model of Alzheimer's disease (Morley, J. F., Brignull, H. R., Weyers, J. J. & Morimoto, R. I. The threshold for polyglutamine-expansion protein aggregation and cellular toxicity is dynamic and influenced by aging in Caenorhabditis elegans. Proc Natl Acad Sci USA 99, 10417-10422 (2002); Florez-McClure, M. L., Hohsfield, L. A., Fonte, G., Bealor, M. T. & Link, C. D. Decreased insulin-receptor signaling promotes the autophagic degradation of betaamyloid peptide in C. elegans. Autophagy 3, 569-580, (2007)) was investigated.

### Materials and Methods

Animals expressing the human A-beta-42 fragment of the APP protein in their muscle were subjected to control or xpo-1 RNAi. Paralyzed animals were quantified as a function of time.

### Results

As shown in FIG. 2F, inhibiting xpo-1 has a remarkable effect on the clearance of Aβ42 peptide in a C. elegans model of Alzheimer's disease.

### Example 4

This example shows that a pharmacological approach to inhibiting xpo-1 is also effective for inducing nuclear localization of HLH-30 and extending lifespans in C. elegans.

### Materials and Methods

Selenixor (KPT-330, Karyopharm), a selective inhibitor of XPO1, was used to determine whether chemical inhibition of nuclear export via XPO-1 mimics the gene silencing (RNAi) approach that generated an autophagy-inducing, lifespan-extending phenotype in nematodes as shown in Examples 1 and 2.

Animals were fed OP50 E. coli with 0.2% of DMSO (vehicle control) or 0.2% DMSO with KPT-330 at 0.2 mM. Nuclear localization of the HLH-30-GFP reporter and survival was measured.

l Lifespan analysis in Drosophila: Measurement of lifespan (25° C.) in dsodH71Y was performed as previously described in the art (Sahin et al., 2017, Human SOD1 ALS Mutations in a Drosophila Knock-In Model Cause Severe Phenotypes and Reveal Dosage-Sensitive Gain- and Loss-of-Function Components. Genetics 205, 707-723). The software Stata 13.0 was used to perform Mantel-Cox log rank statistical analyses.

### Results

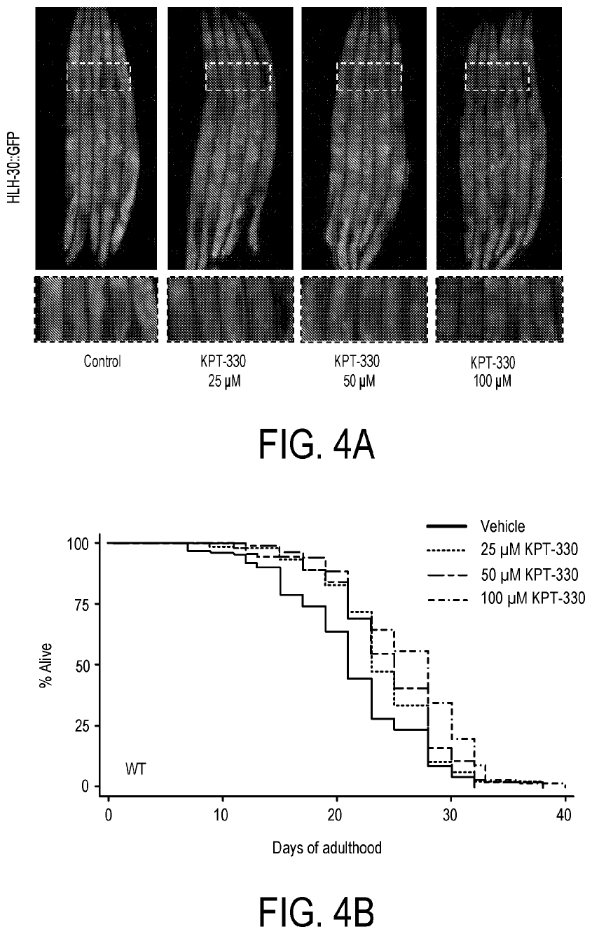

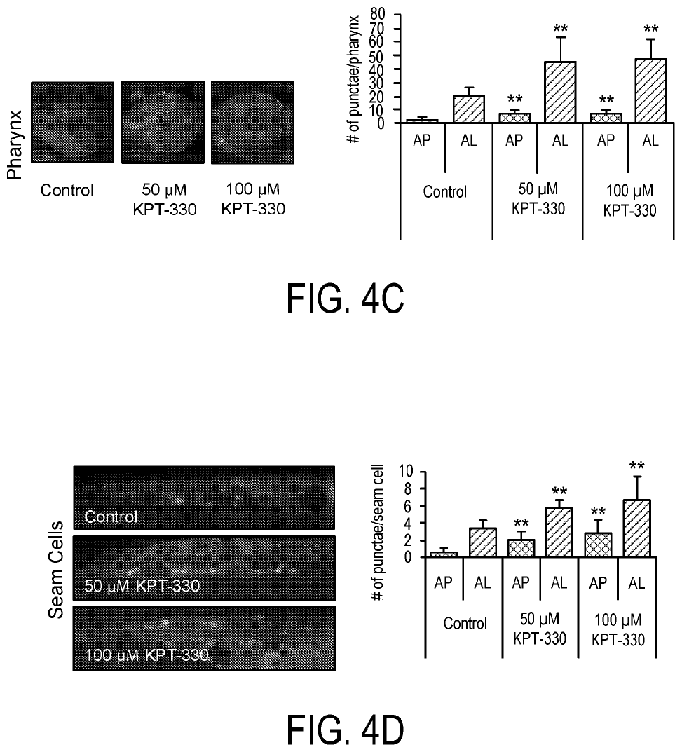

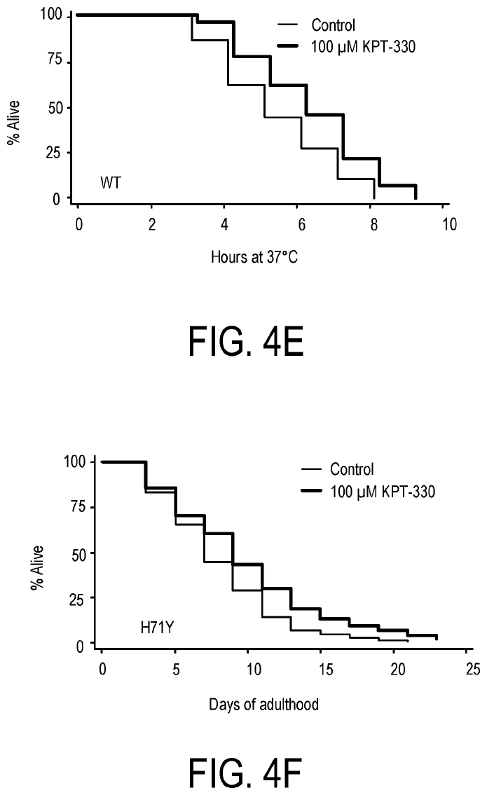

As shown in FIG. 4A, growing nematodes on plates containing 2 mM KPT-330 led to nuclear localization of HLH-30 (fused to GFP). In the results depicted in FIG. 4A, a 2 mM concentration was used because of its clear effect on HLH-30, but lower concentration of KPT-330 also led to nuclear localization of HLH-30 (data not shown). It was also found that treating animals with KPT-330 led to a significant lifespan extension (FIG. 4B). These results support that reducing xpo-1 genetically (FIG. 3A) or pharmacologically (FIG. 4B) activates the nuclear localization of HLH-30 and extends lifespan.

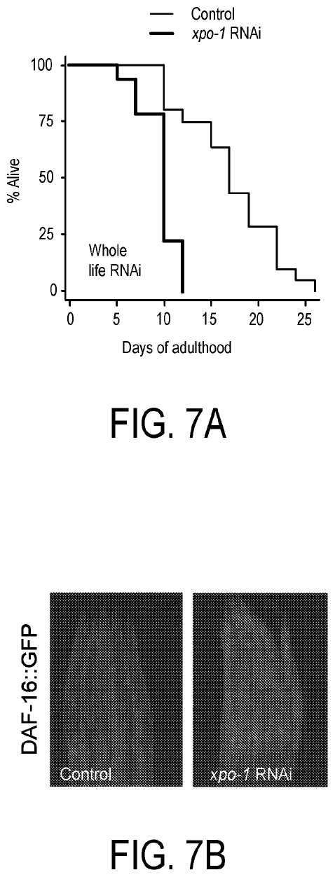

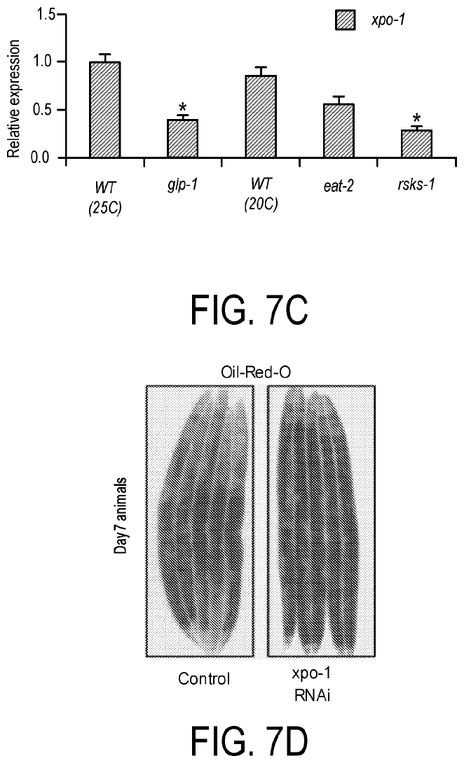

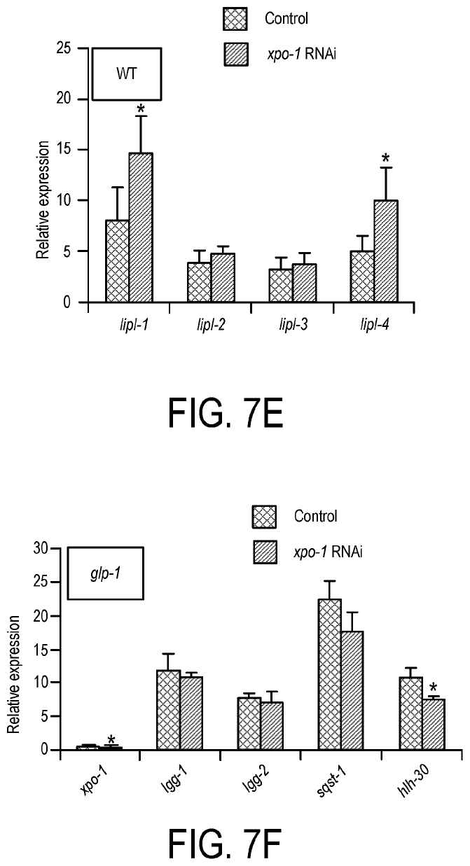

Since elevated autophagy is linked to lifespan extension (Lapierre et al., (2015). Transcriptional and epigenetic regulation of autophagy in aging. Autophagy 11, 867-880), the effects of reducing xpo-1 on life expectancy was further examined. Silencing xpo-1 in adult wild-type animals led to a significant lifespan extension (FIG. 3A and Table 3). Interestingly, xpo-1 displays antagonistic pleiotropy (Kirkwood and Rose, (1991). Evolution of senescence: late survival sacrificed for reproduction. Philos Trans R Soc Lond B Biol Sci 332, 15-24) as whole-life xpo-1 knockdown decreased lifespan (FIG. 7A and Table 3), highlighting developmental roles for XPO-1 followed by a pro-aging impact in adulthood.

TABLE 3
Lifespan analyses of animals treated with xpo-1 RNAi. Details of lifespan of animals fed
control bacteria or bacteria expressing RNAi against xpo-1. Mean lifespan is displayed
in days. Heat stress assay were performed at Day 5 of adulthood (after 5 days of treatment)
and survival is reported in hours (h.). Adult lifespan was assayed at 20° C. Change
in lifespan between control and xpo-1 RNAi is displayed as % difference in mean lifespan.
Corresponding % difference between control and xpo-1 RNAi in wild-type animals is reported
in brackets. Mantel-Cox log rank statistical analyses were performed using Stata 13.0.

| | xpo-1 RNAi | | Control RNAi | | | | |
|---|---|---|---|---|---|---|---|
| | Mean Lifespan | Events | Mean Lifespan | Events | % | | P |
| Strains | (days) | Observed | (days) | Observed | Difference | | Value |
| N2 | 20.7 | 88/100 | 17.9 | 69/100 | 15.6 | | <0.0001 |
| Wild-type (WT) | 23.0 | 87/100 | 17.6 | 68/100 | 30.7 | | <0.0001 |
| | 19.9 | 68/100 | 17.7 | 49/100 | 12.4 | | 0.0156 |
| | 20.9 | 88/100 | 17.9 | 80/100 | 16.8 | | <0.0001 |
| | 18.8 | 48/100 | 14.4 | 67/100 | 30.6 | | <0.0001 |
| | 20.0 | 65/100 | 15.5 | 68/100 | 28.9 | | <0.0001 |
| | 18.1 | 69/100 | 15.0 | 52/100 | 20.7 | | 0.0084 |
| | 16.7 | 78/100 | 14.2 | 68/100 | 17.6 | | 0.0138 |
| | 22.7 | 82/100 | 17.6 | 77/100 | 29.0 | | <0.0001 |
| | 24.0 | 77/100 | 18.0 | 52/100 | 33.3 | | <0.0001 |
| | 22.0 | 80/100 | 15.3 | 56/100 | 43.3 | | <0.0001 |
| | 21.6 | 60/100 | 17.7 | 61/100 | 22.0 | | 0.0002 |
| | 22.0 | 66/100 | 15.7 | 61/100 | 40.1 | | <0.0001 |
| | 20.0 | 81/100 | 16.4 | 73/100 | 21.9 | | <0.0001 |
| CF1037 | 13.0 | 89/100 | 15.0 | 80/100 | −13.3 | (15.6) | 0.0003 |
| daf-16 (mu86) | 13.5 | 90/100 | 13.8 | 91/100 | −2.2 | (30.7) | 0.2635 |
| | 12.8 | 53/100 | 14.6 | 58/100 | −12.3 | (12.4) | 0.0004 |
| LRL1 | 15.4 | 94/100 | 16.3 | 71/100 | −5.5 | (15.6) | 0.004 |
| hlh-30 (tm1978) | 14.8 | 94/100 | 14.4 | 82/100 | 2.8 | (30.7) | 0.7513 |
| | 16.3 | 78/100 | 15.6 | 66/100 | 4.5 | (12.4) | 0.3033 |
| LRL9 | 15.2 | 73/100 | 15.8 | 81/100 | −3.8 | (15.6) | 0.9787 |
| atg-7 (bp411) | 15.6 | 96/100 | 14.2 | 72/100 | 9.9 | (30.6) | 0.0313 |
| | 18.1 | 79/100 | 16.5 | 98/100 | −8.8 | (16.8) | 0.01 |
| VC893 | 14.8 | 71/100 | 15.2 | 73/100 | −2.6 | (15.6) | 0.7583 |
| atg-18 (gk378) | 15.4 | 80/100 | 16.0 | 80/100 | −3.8 | (30.6) | 0.4959 |
| | 14.7 | 90/100 | 14.9 | 78/100 | −1.3 | (16.8) | 0.9485 |
| AA292 | 16.4 | 85/100 | 14.4 | 82/100 | 13.9 | (15.6) | 0.0006 |
| daf-36 (k114) | 15.6 | 78/100 | 12.7 | 75/100 | 22.8 | (29.0) | 0.0002 |
| | 15.2 | 73/100 | 13.1 | 73/100 | 16.0 | (43.8) | 0.0292 |
| CF1903 | 14.9 | 76/100 | 18.6 | 70/100 | −20.1 | (28.9) | 0.0011 |
| glp-1 (e2144) | 15.6 | 92/100 | 17.1 | 83/100 | −8.8 | (20.7) | 0.2834 |
| | 13.7 | 92/100 | 14.9 | 85/100 | −8.1 | (17.6) | 0.1569 |
| MAH95 | 19.9 | 84/100 | 19.9 | 52/100 | 0.0 | (28.9) | 0.7297 |
| eat-2 (ad1116) | 28.9 | 74/100 | 20.7 | 38/100 | −8.7 | (20.7) | 0.2838 |
| | 19.2 | 75/100 | 18.3 | 70/100 | 4.9 | (17.6) | 0.4372 |
| VB633 | 18.0 | 77/100 | 18.9 | 68/100 | −4.5 | (28.9) | 0.4634 |
| rsks-1 (sv31) | 19.1 | 70/100 | 19.5 | 77/100 | −2.1 | (17.6) | 0.9765 |

| | | | | | | |
|---|---|---|---|---|---|---|
| N2 (WT) | 9.7 | 76/100 | 17.2 | 82/100 | −43.6 | <0.0001 |
| Whole-life RNAi | 10.4 | 80/100 | 16.8 | 73/100 | −38.1 | <0.0001 |
| N2 (WT) | 4.9 h. | 173/200 | 3.7 h. | 154/200 | 32.4 | <0.0001 |
| Heat Stress | 5.9 h. | 130/200 | 4.1 h. | 114/200 | 43.9 | <0.0001 |

Next, the contribution of autophagy-related transcription factors and proteins in lifespan extension was evaluated. Subjecting hlh-30(tm1978) or daf-16(mu86) mutants to RNAi against xpo-1 had no effect on their lifespan (FIG. 3B and FIG. 3C; Table 3). DAF-16 was also found localized in the nucleus of animals subjected to xpo-1 knockdown, suggesting potential co-regulation with HLH-30 (FIG. 7B). Silencing xpo-1 in autophagy defective mutants, atg-18(gk378) and atg-7(bp411), did not affect lifespan (FIG. 3D, FIG. 3E, and Table 3). Thus, without being bound to theory, autophagy is required for the longevity effect associated with xpo-1 silencing. Using daf-36(k114) mutants (Rottiers et al., (2006). Hormonal control of C. elegans dauer formation and life span by a Rieske-like oxygenase. Dev Cell 10, 473-482), it was found that lifespan extension from xpo-1 RNAi was not dependent on gonadal signaling (FIG. 3F and Table 3).

To explore the similarities between xpo-1 silencing and established long-lived models, xpo-1 in dietary-restricted eat-2(ad1116), germline-less glp-1(e2144), and protein synthesis rsks-1(sv31) mutants was silenced. Reducing xpo-1 expression in these mutants had no additive effect on their lifespan suggesting mechanistic overlap (FIG. 3G, FIG. 3H, FIG. 3I, and Table 3). Notably, xpo-1 mRNA levels were low in these three established longevity models (FIG. 7C). As observed in several long-lived animals, silencing xpo-1 resulted in elevated lipid storage (FIG. 7D) (Lapierre et al., (2013). Autophagy genes are required for normal lipid levels in C. elegans. Autophagy 9, 278-286; Seah et al., (2016). Autophagy-mediated longevity is modulated by lipoprotein biogenesis. Autophagy 12, 261-272) and increased lysosomal lipase gene expression (FIG. 7E) (Lapierre et al., (2011). Autophagy and lipid metabolism coordinately modulate life span in germline-less C. elegans. Curr Biol 21, 1507-1514; O'Rourke and Ruvkun, (2013). MXL-3 and HLH-30 transcriptionally link lipolysis and autophagy to nutrient availability. Nat Cell Biol 15, 668-676; Seah et al., (2016). Autophagy-mediated longevity is modulated by lipoprotein biogenesis. Autophagy 12, 261-272.; Wang et al., (2008). Fat metabolism links germline stem cells and longevity in C. elegans. Science 322, 957-960). Autophagy gene expression (FIG. 2B) was not further enhanced in glp-1(e2144) animals subjected to xpo-1 RNAi (FIG. 7F). Taken together, these data indicate that lifespan extension from xpo-1 silencing mechanistically mimics established longevity models and relies on the activity of HLH-30/TFEB, DAF-16/FOXO as well as functional autophagy.

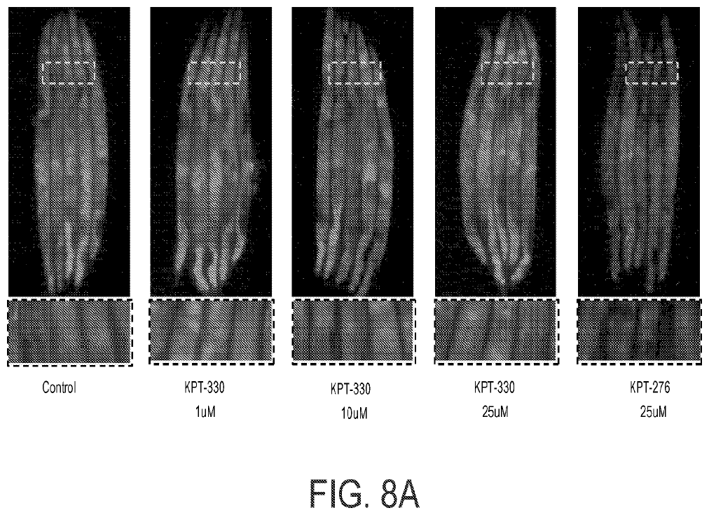

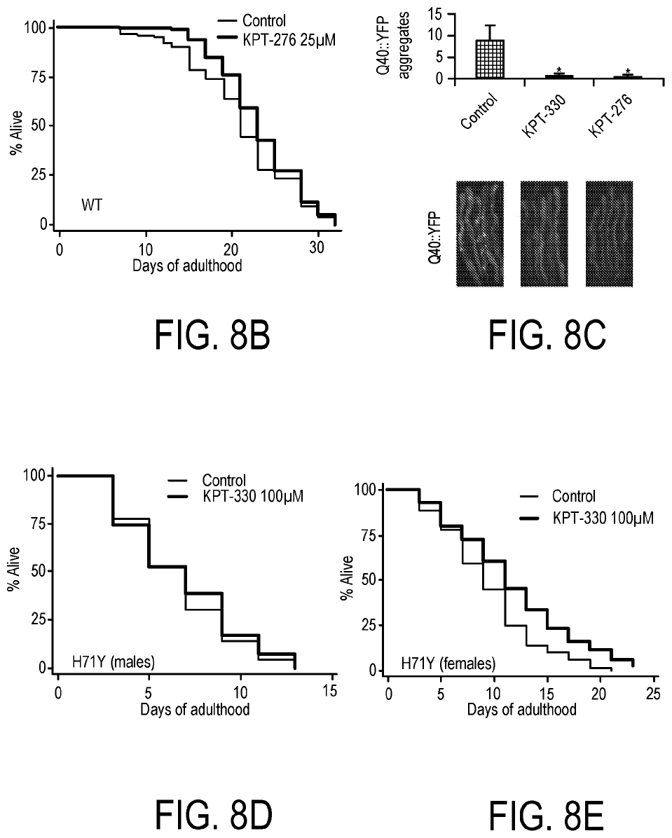

The effect of XPO-1/XPO1 inhibition by KPT on autophagy and lifespan in C. elegans was further examined. Day 1 adults subjected to different concentrations (25, 50 and 100 μM) of KPT-330 for 72 hours displayed nuclear localization of HLH-30 (FIG. 4A). These concentrations fell within the typical range used in drug screening studies in C. elegans (Benedetti et al., (2008). Compounds that confer thermal stress resistance and extended lifespan. Exp Gerontol 43, 882-891; Ye et al., (2014). A pharmacological network for lifespan extension in Caenorhabditis elegans. Aging Cell 13, 206-215). Nuclear localization of HLH-30::GFP was not detected at concentrations below 25 μM (FIG. 8A). A significant increase in the lifespan of animals treated with KPT-330 was observed compared to vehicle-treated controls (FIG. 4B and Table 4). Similarly, nuclear enrichment of HLH-30 and lifespan extension were observed in animals treated with KPT-276 at 25 μM (FIG. 8A, FIG. 8B, and Table 4).

TABLE 4
Lifespan analyses of animals treated with XPO1 inhibitors. Details of lifespan of animals incubated
with OP50-seeded plates with DMSO (0.1%), KPT-330 or KPT-276. Nematode lifespan was measured at 20°
C. and fly lifespan was performed at 25° C. Mean lifespan is displayed in days. Heat stress assay
were performed at Day 5 of adulthood (after 5 days of treatment) and survival is reported in hours
(h.). Variation in lifespan between vehicle control and compound treatment is displayed as % difference
in mean lifespan. Mantel-Cox log rank statistical analyses were performed using Stata 13.0.

| | | Drug treatment | | Vehicle control | | | |
|---|---|---|---|---|---|---|---|
| | | Mean Lifespan | Events | Mean Lifespan | Events | % | P |
| Strains | Treatment | (days) | Observed | (days) | Observed | Difference | Value |
| C. elegans | | | | | | | |
| N2 | KPT-330 | | | | | | |
| Wild-type (WT) | 25 μM | 21.7 | 93/125 | 19.3 | 94/125 | 12.4 | <0.0001 |
| | 50 μM | 21.2 | 98/125 | 19.3 | 94/125 | 9.8 | <0.0001 |
| | 100 μM | 22 | 69/125 | 19.3 | 94/125 | 14 | <0.0001 |
| N2 | KPT-330 | | | | | | |
| Wild-type (WT) | 25 μM | 24 | 136/250 | 21.2 | 114/250 | 13.2 | 0.0005 |
| | 50 μM | 24.8 | 138/250 | 21.2 | 114/250 | 17 | <0.0001 |
| | 100 μM | 25.8 | 84/250 | 21.2 | 114/250 | 21.7 | <0.0001 |
| N2 | KPT-276 | | | | | | |
| Wild-type (WT) | 25 μM | 23.2 | 124/250 | 21.2 | 114/250 | 9.4 | 0.0293 |
| N2 | KPT-330 (100 μM) | 6.4 h. | 75/125 | 5.4 h. | 78/125 | 18.5 | <0.0001 |
| Wild-type (WT) | Heat Stress | 6.2 h. | 109/125 | 4.6 h. | 90/125 | 34.7 | <0.0001 |
| D. melanogaster | | | | | | | |
| H71Y | KPT-330 (100 μM) | | | | | | |
| Sod-1 (dsod) | Both sexes | 9.8 | 312/324 | 7.9 | 376/387 | 24.0 | <0.0001 |
| | Females | 12 | 171/180 | 9.5 | 182/187 | 26.3 | <0.0001 |
| | Males | 6.8 | 141/144 | 6.5 | 194/200 | 4.6 | 0.3116 |
| H71Y | KPT-330 (100 μM) | | | | | | |
| Sod-1 (dsod) | Both sexes | 13.5 | 152/152 | 11.4 | 193/193 | 18.4 | 0.0160 |
| | Females | 16.9 | 103/103 | 13.3 | 141/141 | 27.1 | 0.0013 |
| | Males | 6 | 49/49 | 6.4 | 52/52 | −6.3 | 0.3530 |

Animals expressing mCherry::GFP::LGG-1 were treated with vehicle control or KPT-330 (50 or 100 μM) and visualized after 48 hours. Autophagy was enhanced in pharynx and hypodermal seam cells of drug-treated animals (FIG. 4C and FIG. 4D). Autophagic increases translated into improved resistance to heat (FIG. 4E) and a reduction in the formation of polyQ-containing (Q40::YFP) aggregates (Mohri-Shiomi and Garsin, (2008). Insulin signaling and the heat shock response modulate protein homeostasis in the Caenorhabditis elegans intestine during infection. J Biol Chem 283, 194-201) (FIG. 8C). Since KPT-330 was able to extend lifespan in nematodes, we sought to analyze its effect on the lifespan of a Sod1-based neurodegenerative model of Amyotrophic Lateral Sclerosis (ALS) in flies (dsod^{H71Y}) (Sahin et al., (2017). Human SOD1 ALS Mutations in a Drosophila Knock-In Model Cause Severe Phenotypes and Reveal Dosage-Sensitive Gain- and Loss-of-Function Components. Genetics 205, 707-723). Feeding KPT-330 (100 μM) to dsod^{H71Y} flies during adulthood had a beneficial impact on the overall survivorship compared to control (FIG. 4F). Specifically, the lifespan of females, but not of males, was extended significantly (FIG. 8 D, FIG. 8E, and Table 4), suggesting sex-specific benefits associated with the inhibition of Embargoed/XPO1 and supporting a conserved role for XPO1 in the regulation of proteostasis. Altogether, these data demonstrate that pharmacological inhibition of XPO-1 mimics the effect of xpo-1 RNAi and protects against neurodegeneration in flies.

### Example 5

This Example demonstrates that XPO1 inhibition leads to TFEB nuclear enrichment and autophagy enhancement.

### Materials and Methods

Cell culture: HeLa cells (ATCC) and HeLa cells expressing TFEB-GFP (S. Ferguson lab) were grown in 6-well plates on a coverslip for 24 hours in DMEM High Glucose (Genesee Scientific) containing 2 mM L-Glutamine, 1% Penicillin-Strep and 10% Fetal Bovine Serum (GenClone). For RNAi experiments, both HeLa cell lines were transfected with Silencer Select siRNA against XPO1 (5 nM) or TFEB (10 nM) or negative control siRNA #2 (Thermo Fisher Scientific) with RNAi Max (Thermo Fisher Scientific) and Opti-MEM (Gibco) for 48 hours. Cells were incubated for another 6 hours with DMSO 0.1%, Torin 1 (204), KPT-330, KPT-276, KPT-185 or KPT-335 (1 μM) (Selleckchem). For Lysotracker analysis, cells on coverslips were previously grown with vehicle or compounds (including GSK-3β inhibitor VIII or Leptomycin b (10 nM) for 6 hours followed by a one hour incubation with 100 nM of Lysotracker Red DND-99 (Thermo Fisher Scientific) and live cells were imaged. Coverslips were mounted for imaging onto slides and imaged with a Zeiss Axiovert 200M Fluorescent Microscope. Lysotracker signal was quantified by measuring signal intensity over background of individual cells using ImageJ. For imaging TFEB-GFP signal, cells were fixed in methanol. Nuclear localization was quantified by counting cells that had higher GFP intensity in the nucleus compared to the cytoplasm measured using ImageJ.

Immunoblotting: HeLa cells were plated and grown for 24 hours. Thereafter, cells were treated for 24 hours with control or compounds. After 24 hours, cells were collected with a SDS-RIPA lysis buffer (50 mM Tris-HCl, 150 mM NaCl and 1 mM EDTA) including 2% SDS, 1 mM PMSF and Complete ULTRA Protease Inhibitors (Roche). Protein content was measured using the DC Protein Assay (Bio-Rad). 30 μg of proteins were loaded onto 4-15% TGX gels (Bio-Rad) and resolved by electrophoresis. Proteins were transferred using Trans Blot Turbo Transfer System (Bio-Rad) onto nitrocellulose membrane (Bio-Rad). Immunoblotting were conducted using antibodies against Tubulin (ab6160, Abcam), LC3 (ab51520, Abcam), mTOR (2983S, Cell Signaling), phospho-mTOR (5536S, Cell Signaling), p70 S6 Kinase (2708S, Cell Signaling), phosphor-p70 S6 Kinase (9205S, Cell Signaling), XPO1 (sc74454, Santacruz), p62 (ab56416, Abcam). Proteins were visualized with ECL reagents (SuperSignal West Pico and West Femto, Pierce) using a ChemiDoc Imaging System (Bio-Rad).

### Results

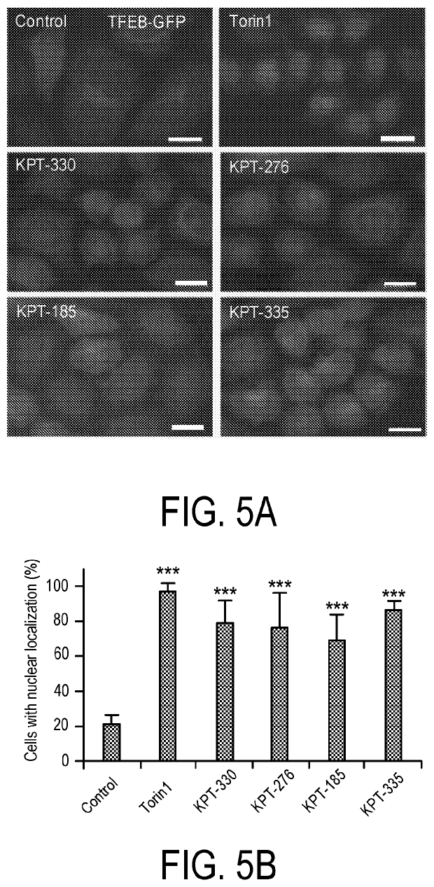

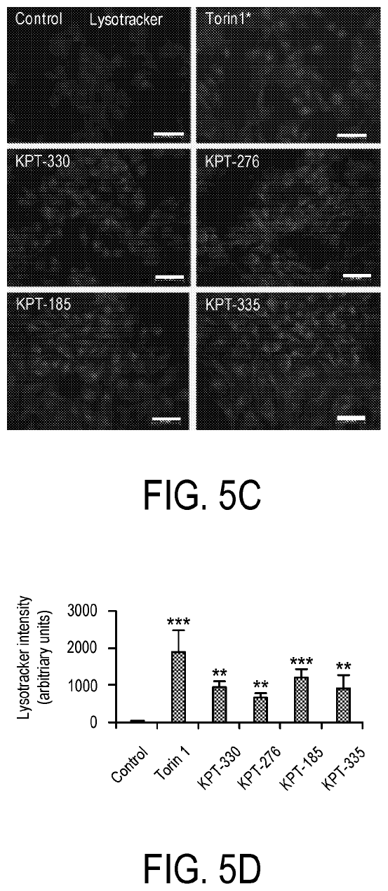

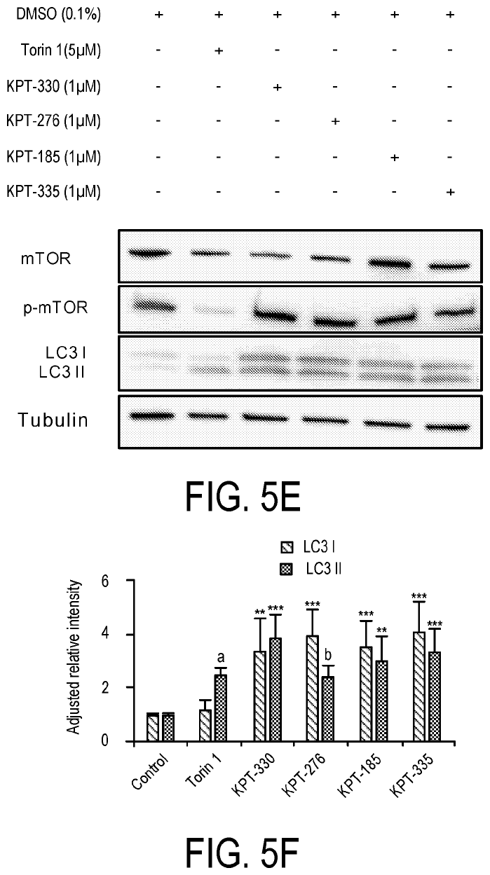

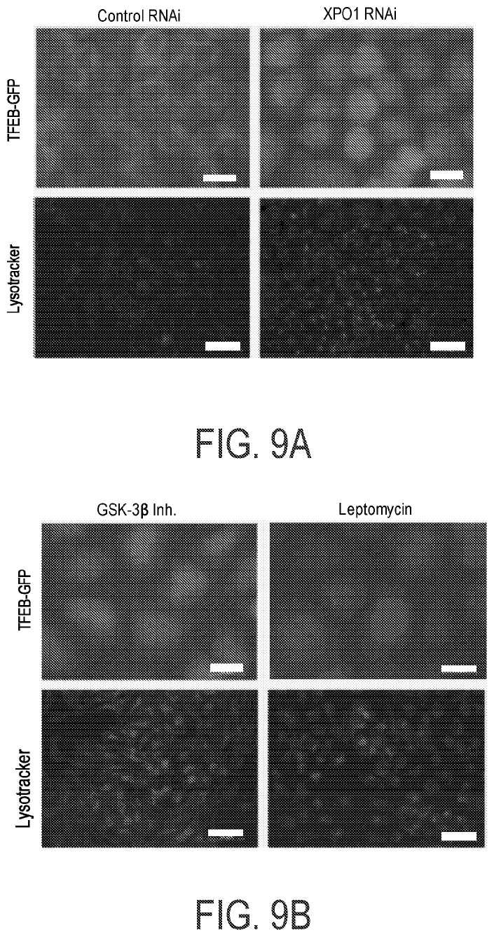

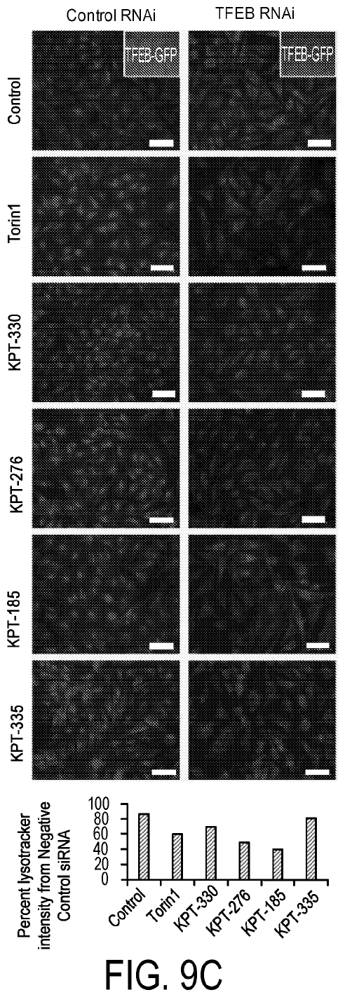

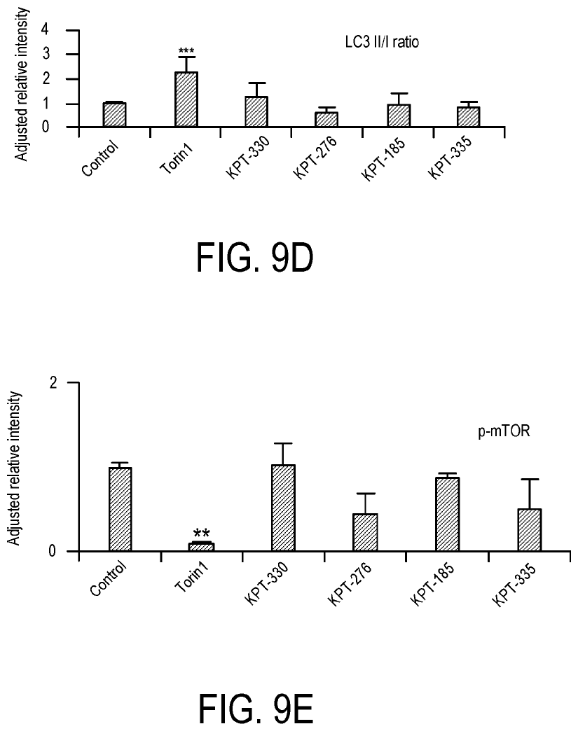

In order to determine whether or not the effect of XPO-1/XPO1 inhibition on HLH-30/TFEB and autophagy is conserved in humans, the effect of XPO1 inhibitors in HeLa cells was analyzed. Incubating TFEB-GFP-expressing HeLa cells (Roczniak-Ferguson et al., (2012). The transcription factor TFEB links mTORC1 signaling to transcriptional control of lysosome homeostasis. Sci Signal 5, ra42) with various selective inhibitors of nuclear export (KPT-330, KPT-276, KPT-185, KPT-335) showed a marked increase in nuclear localization of TFEB (FIG. 5A and FIG. 5B). Similar observations were obtained when cells were incubated with mTOR inhibitor Torin 1, with RNAi against XPO1, with GSK-3β inhibitor VIII or with Leptomycin B (FIG. 5A, FIG. 5B, FIG. 9A and FIG. 9B). Enhanced nuclear localization of TFEB was accompanied by marked increase in red lysotracker signal, corresponding to an increased presence of acidic lysosomal compartments (FIG. 5C). Enhanced lysotracker signal from mTOR or XPO1 inhibition required TFEB (FIG. 9C). Levels of both forms of LC3 (I and II) were increased under XPO1 inhibition suggesting an overall upregulation of autophagosome formation and maturation, but their ratio did not significantly differ from control (FIG. 5D and FIG. 9D). In contrast to Torin 1, the effect associated with XPO1 inhibition did not rely on reducing mTOR signaling as phosphorylation of mTOR was not affected by XPO1 inhibition (FIG. 5D and FIG. 9E). Taken together, these results demonstrate a new and conserved strategy to enhance autophagy and lysosomal function via TFEB nuclear enrichment by inhibiting the conserved nuclear export protein XPO1.

SEQUENCES

Human Exportin-1 protein sequence UniProtKB-O14980 (XPO1_HUMAN) (SEQ ID
NO: 1)
Bold and underlined = Importin N-terminal domain
MPAIMTMLADHAARQLLDFSQKLDINLLDNVVNCLYHGEGAQQRMAQEVLTHLKEHPDAW
TRVDTILEFSQNMNTKYYGLQILENVIKTRWKILPRNQCEGIKKYVVGLIIKTSSDPTCV
EKEKVYIGKLNMILVQILKQEWPKHWPTFISDIVGASRTSESLCQNNMVILKLLSEEVFD
FSSGQITQVKSKHLKDSMCNEFSQIFQLCQFVMENSQNAPLVHATLETLLRFLNWIPLGY
IFETKLISTLIYKFLNVPMFRNVSLKCLTEIAGVSVSQYEEQFVTLFTLTMMQLKQMLPL
NTNIRLAYSNGKDDEQNFIQNLSLFLCTFLKEHDQLIEKRLNLRETLMEALHYMLLVSEV
EETEIFKICLEYWNHLAAELYRESPFSTSASPLLSGSQHFDVPPRRQLYLPMLFKVRLLM
VSRMAKPEEVLVVENDQGEVVREFMKDTDSINLYKNMRETLVYLTHLDYVDTERIMTEKL
HNQVNGTEWSWKNLNTLCWAIGSISGAMHEEDEKRFLVTVIKDLLGLCEQKRGKDNKAII
ASNIMYIVGQYPRFLRAHWKFLKTVVNKLFEFMHETHDGVQDMACDTFIKIAQKCRRHFV
QVQVGEVMPFIDEILNNINTIICDLQPQQVHTFYEAVGYMIGAQTDQTVQEHLIEKYMLL
PNQVWDSIIQQATKNVDILKDPETVKQLGSILKTNVRACKAVGHPFVIQLGRIYLDMLNV
YKCLSENISAAIQANGEMVTKQPLIRSMRTVKRETLKLISGWVSRSNDPQMVAENFVPPL
LDAVLIDYQRNVPAAREPEVLSTMAIIVNKLGGHITAEIPQIFDAVFECTLNMINKDFEE
YPEHRTNFFLLLQAVNSHCFPAFLAIPPTQFKLVLDSIIWAFKHTMRNVADTGLQILFTL
LQNVAQEEAAAQSFYQTYFCDILQHIFSVVTDTSHTAGLTMHASILAYMFNLVEEGKIST
SLNPGNPVNNQIFLQEYVANLLKSAFPHLQDAQVKLFVTGLFSLNQDIPAFKEHLRDFLV
QIKEFAGEDTSDLFLEEREIALRQADEEKHKRQMSVPGIFNPHEIPEEMCD
Human Exportin-1 mRNA sequence NCBI Reference Sequence: NM_003400.3
(SEQ ID NO: 2)
Bold and underlined = Start and Stop codons

| | |
|---|---|
| 1 | aggaaggcca cgtccccggg gagggacgcc cgactcgatg gtctgcgcag ggcccgtcgg |
| 1 | caaccggttc cgagtttgag gcactaggag gagggggaga agcggctgca gcggccgcgg |
| 61 | caggagcagc gggagctaca gcatcagcaa gagcaacagt agctacagcc ccggcggcgg |
| 121 | tgcctgttcc agtctttgct gctgcagtcc gtgcaaccac ccagaggggg aggggggaac |
| 181 | caccagtcgc tgaggaacaa gagaaggggg gaaagtttag gcgagccttg gggggggggg |
| 241 | ggccagcgcc ggagccgcgt gagagaggga gccgtgtttt ggtagggggg agtcggactg |
| 301 | caactggcag cagagcgtct ccccggccgt gtggactcta caccccctac tcctgccgct |
| 361 | tctgctgctg cctgtggctg gagggtcccc ctggggctga atctttggga cttgaccccg |
| 421 | ttccctcccc cttccctcac tccccagccg ggcgggagca tttattcccc agattaattc |
| 481 | cccttttggg ggggggcggg tgtgtgtgtg tgtgtgtgtg tgtgtgtgtg ttgggggaag |
| 541 | cgtccctgaa atagtaaata ttattgagct ctttttgccc ttttcctgtc cgttttttta |
| 601 | atttcctttt ttgaggtggg aaaactgaaa cccaccttga ttcgtcccct ctcccccctc |
| 661 | cccaccttcc ctcgccctaa tcccccaacg aggaaggaag gagcagttgg ttcaatctct |
| 721 | ggtaatctAT Gccagcaatt atgacaatgt tagcagacca tgcagctcgt cagctgcttg |
| 781 | atttcagcca aaaactggat atcaacttat tagataatgt ggtgaattgc ttataccatg |
| 841 | gagaaggagc ccagcaaaga atggctcaag aagtactgac acatttaaag gagcatcctg |
| 901 | atgcttggac aagagtcgac acaattttgg aattttctca gaatatgaat acgaaatact |
| 961 | atggactaca aattttggaa aatgtgataa aaacaaggtg gaagattctt ccaaggaacc |
| 1021 | agtgcgaagg aataaaaaaa tacgttgttg gcctcattat caagacgtca tctgacccaa |
| 1081 | cttgtgtaga gaaagaaaag gtgtatatcg gaaaattaaa tatgatcctt gttcagatac |
| 1141 | tgaaacaaga atggcccaaa cattggccaa cttttatcag tgatattgtt ggagcaagta |
| 1201 | ggaccagcga aagtctctgt caaaataata tggtgattct taaactcttg agtgaagaag |
| 1261 | tatttgattt ctctagtgga cagataaccc aagtcaaatc taagcattta aaagacagca |
| 1321 | tgtgcaatga attctcacag atatttcaac tgtgtcagtt tgtaatggaa aattctcaaa |
| 1381 | atgctccact tgtacatgca accttggaaa cattgctcag atttctgaac tggattcccc |
| 1441 | tgggatatat ttttgagacc aaattaatca gcacattgat ttataagttc ctgaatgttc |
| 1501 | caatgtttcg aaatgtctct ctgaagtgcc tcactgagat tgctggtgtg agtgtaagcc |
| 1561 | aatatgaaga acaatttgta acactattta ctctgacaat gatgcaacta aagcagatgc |
| 1621 | ttcctttaaa taccaatatt cgacttgcgt actcaaatgg aaaagatgat gaacagaact |
| 1681 | tcattcaaaa tctcagtttg tttctctgca cctttcttaa ggaacatgat caacttatag |
| 1741 | aaaaaagatt aaatctcagg gaaactctta tggaggccct tcattatatg ttgttggtat |
| 1801 | ctgaagtaga agaaactgaa atctttaaaa tttgtcttga atactggaat catttggctg |
| 1861 | ctgaactcta tagagagagt ccattctcta catctgcctc tccgttgctt tctggaagtc |
| 1921 | aacattttga tgttcctccc aggagacagc tatatttgcc catgttattc aaggtccgtt |
| 1981 | tattaatggt tagtcgaatg gctaaaccag aggaagtatt ggttgtagag aatgatcaag |
| 2041 | gagaagttgt gagagaattc atgaaggata cagattccat aaatttgtat aagaatatga |
| 2101 | gggaaacatt ggtttatctt actcatctgg attatgtaga tacagaaaga ataatgacag |
| 2161 | agaagcttca caatcaagtg aatggtacag agtggtcatg gaaaaatttg aatacattgt |
| 2221 | gttgggcaat aggctccatt agtggagcaa tgcatgaaga ggacgaaaaa cgatttcttg |
| 2281 | ttactgttat aaaggatcta ttaggattat gtgaacagaa aagaggcaaa gataataaag |
| 2341 | ctattattgc atcaaatatc atgtacatag taggtcaata cccacgtttt ttgagagctc |
| 2401 | actggaaatt tctgaagact gtagttaaca agctgttcga attcatgcat gagacccatg |
| 2461 | atggagtcca ggatatggct tgtgatactt tcattaaaat agcccaaaaa tgccgcaggc |
| 2521 | atttcgttca ggttcaggtt ggagaagtga tgccatttat tgatgaaatt ttgaacaaca |
| 2581 | ttaacactat tatttgtgat cttcagcctc aacaggttca tacgttttat gaagctgtgg |
| 2641 | ggtacatgat tggtgcacaa acagatcaaa cagtacaaga acacttgata gaaaagtaca |
| 2701 | tgttactccc taatcaagtg tgggatagta taatccagca ggcaaccaaa aatgtggata |
| 2761 | tactgaaaga tcctgaaaca gtcaagcagc ttggtagcat tttgaaaaca aatgtgagag |
| 2821 | cctgcaaagc tgttggacac ccctttgtaa ttcagcttgg aagaatttat ttagatatgc |
| 2881 | ttaatgtata caagtgcctc agtgaaaata tttctgcagc tatccaagct aatggtgaaa |
| 2941 | tggttacaaa gcaaccattg attagaagta tgcgaactgt aaaaagggaa actttaaagt |
| 3001 | taatatctgg ttgggtgagc cgatccaatg atccacagat ggtcgctgaa aattttgttc |
| 3061 | cccctctgtt ggatgcagtt ctcattgatt atcagagaaa tgtcccagct gctagagaac |
| 3121 | cagaagtgct tagtactatg gccataattg tcaacaagtt agggggacat ataacagctg |
| 3181 | aaatacctca aatatttgat gctgtttttg aatgcacatt gaatatgata aataaggact |
| 3241 | ttgaagaata tcctgaacat agaacgaact ttttcttact acttcaggct gtcaattctc |
| 3301 | attgtttccc agcattcctt gctattccac ctacacagtt taaacttgtt ttggattcca |
| 3361 | tcatttgggc tttcaaacat actatgagga atgtcgcaga tacgggctta cagatacttt |
| 3421 | ttacactctt acaaaatgtt gcacaagaag aagctgcagc tcagagtttt tatcaaactt |
| 3481 | atttttgtga tattctccag catatctttt ctgttgtgac agacacttca catactgctg |
| 3541 | gtttaacaat gcatgcatca attcttgcat atatgtttaa tttggttgaa gaaggaaaaa |
| 3601 | taagtacatc attaaatcct ggaaatccag ttaacaacca aatctttctt caggaatatg |
| 3661 | tggctaatct ccttaagtcg gccttccctc acctacaaga tgctcaagta aagctctttg |
| 3721 | tgacagggct tttcagctta aatcaagata ttcctgcttt caaggaacat ttaagagatt |
| 3781 | tcctagttca aataaaggaa tttgcaggtg aagacacttc tgatttgttt ttggaagaga |
| 3841 | gagaaatagc cctacggcag gctgatgaag agaaacataa acgtcaaatg tctgtccctg |
| 3901 | gcatctttaa tccacatgag attccagaag aaatgtgtga tTAAaatcca aattcatgct |
| 3961 | gttttttttc tctgcaactc gttagcagag gaaaacagca tgtgggtatt tgtcgaccaa |
| 4021 | aatgatgcca atttgtaaat taaaatgtca cctagtggcc ctttttctta tgtgtttttt |
| 4081 | tgtataagaa attttctgtg aaatatcctt ccattgttta agcttttgtt ttggtcatct |
| 4141 | ttatttagtt tgcatgaagt tgaaaattaa ggcattttta aaaattttac ttcatgccca |
| 4201 | tttttgtggc tgggctgggg ggaggaggca aattcgattt gaacatatac ttgtaattct |
| 4261 | aatgcaaaat tatacaattt ttcctgtaaa caataccaat ttttaattag ggagcatttt |
| 4321 | ccttctagtc tatttcagcc tagaagaaaa gataatgagt aaaacaaatt gcgttgttta |
| 4381 | aaggattata gtgctgcatt gtctgaagtt agcacctctt ggactgaatc gtttgtctag |
| 4441 | actacatgta ttacaaagtc tctttggcaa gattgcagca agatcatgtg catatcatcc |
| 4501 | cattgtaaag cgacttcaaa aatatgggaa cacagttagt tatttttaca cagttctttt |
| 4561 | tgtttttgtg tgtgtgtgct gtcgcttgtc gacaacagct ttttgttttc ctcaatgagg |
| 4621 | agtgttgctc atttgtgagc cttcattaac tcgaagtgaa atggttaaaa atatttatcc |
| 4681 | tgttagaata ggctgcatct ttttaacaac tcattaaaaa acaaaacaac tctggctttt |
| 4741 | gagatgactt atactaattt acattgttta ccaagctgta gtgctttaag aacactactt |
| 4801 | aaaaagcaaa ataaacttgg tttacattta |

C. elegans xpo-1 protein sequence UniProtKB-Q23089 (Q23089_CAEEL)
(SEQ ID NO: 3)
Bold and underlined = Importin N-terminal domain
MAVSAMEVLSEAKRQFAQGDRIDVTLLDQVVEIMNRMSGKEQAEANQILMSLKEERDSWT
KVDAILQYSQLNESKYFALQILETVIQHKWKSLPQVQREGIKSYIITKMFELSSDQSVME
QSQLLLHKLNLVLVQIVKQDWPKAWPTFITDIVDSSKNNETVCINNMNILSLLSEEVFDF
GSQNLTQAKEQHLKQQFCGQFQEVFTLCVSILEKCPSNSMVQATLKTLQRFLTWIPVGYV
FETNITELLSENFLSLEVYRVIALQCLTEISQIQVETNDPSYDEKLVKMFCSTMRHISQV
LSLDLDLAAVYKDASDQDQKLISSLAQFLVAFIKEHVHLIEVTDEPLTEAKILMRESHDY
AIQLLLKITLIEEMEVFKVCLDCWCWLTAELYRICPFIQPSTLYGMMSQVREHPRRQLYR
EYLSQLRSTMISRMAKPEEVLIVENDQGEVVREMVKDTDSIALYRNMRETLVYLTHLDNK
DTEVKMTEKLASQVNGGEFSWKNLNRLCWAVGSISGTMVEEDEKRFLVLVIRDLLGLCEQ
KRGKDNKAVIASNIMYVVGQYPRFLRAHWKFLKTVINKLFEFMHETHEGVQDMACDTFIK
ISIKCKRHFVIVQPAENKPFVEEMLENLTGIICDLSHAQVHVFYEAVGHIISAQIDGNLQ
EDLIMKLMDIPNRTWNDIIAAASTNDSVLEEPEMVKSVLNILKTNVAACKSIGSSFVTQL
GNIYSDLLSLYKILSEKVSRAVTTAGEEALKNPLVKTMRAVKREILILLSTFISKNGDAK
LILDSIVPPLFDAVLFDYQKNVPQAREPKVLSLLSILVTQLGSLLCPQVPSILSAVFQCS
IDMINKDMEAFPEHRTNFFELVLSLVQECFPVFMEMPPEDLGTVIDAVVWAFQHTMRNVA
EIGLDILKELLARVSEQDDKIAQPFYKRYYIDLLKHVLAVACDSSQVHVAGLTYYAEVLC
ALFRAPEFSIKVPLNDANPSQPNIDYIYEHIGGNFQAHFDNMNQDQIRIIIKGFFSFNTE
ISSMRNHLRDFLIQIKEHNGEDTSDLYLEEREAEIQQAQQRKRDVPGILKPDEVEDEDMR
C. elegans xpo-1 mRNA sequence NCBI Reference Sequence: NM_171484.5 (SEQ
ID NO: 4)
Bold and underlined = Start and Stop codons

| | |
|---|---|
| 1 | ATGgctgtct cagcaatgga agtgctctct gaagcgaagc gtcaattcgc ccaaggtgac |
| 61 | cgtatcgatg tgactctttt ggatcaagtc gtcgagatta tgaatcgaat gagcggaaaa |
| 121 | gaacaggccg aggctaacca aatcctcatg tcgctcaagg aagaacgcga ttcgtggacc |
| 181 | aaagtcgatg cgattcttca atattcgcag ctaaatgagt ccaagtattt tgcacttcaa |
| 241 | atccttgaga ctgtcatcca acacaaatgg aagtcgttgc cacaagtcca acgtgaagga |
| 301 | atcaagtcgt acatcatcac caaaatgttc gaattgtctt ctgatcagag tgtcatggaa |
| 361 | caaagtcaac tgcttcttca caagttgaac ctcgttttgg ttcaaattgt caaacaagat |
| 421 | tggccaaagg catggccaac attcatcacc gatattgttg attcatcgaa gaacaacgag |
| 481 | accgtttgca tcaacaatat gaatattctg agcttgttga gcgaagaagt atttgatttc |
| 541 | ggatcccaaa acctcaccca agccaaggaa caacatctga aacaacaatt ctgtggacag |
| 601 | ttccaagaag tattcacact gtgtgtcagc attctcgaga aatgtccatc caactctatg |
| 661 | gttcaagcta ccttgaagac tcttcaacga ttcctcacct ggattccagt tggctacgtt |
| 721 | ttcgagacaa acatcacgga attgctgtct gaaaacttcc tttcgcttga agtgtatcgt |
| 781 | gtcatcgctc ttcaatgtct cacggaaatt tcacaaattc aagttgaaac caacgatccc |
| 841 | agctacgacg aaaagcttgt caaaatgttc tgctctacaa tgcgccacat tagccaagtt |
| 901 | ctatctttgg acctcgacct ggctgctgtc tacaaagatg cttccgatca ggatcagaaa |
| 961 | ctcatcagta gtctggctca atttcttgtc gcgttcatca aagagcacgt tcatctgatt |
| 1021 | gaagtaactg atgagccatt aactgaggct aaaattttaa tgcgagaatc tcacgactat |
| 1081 | gctattcaac ttcttttgaa aatcactttg atcgaagaaa tggaggtttt caaagtttgc |
| 1141 | ctcgattgtt ggtgctggtt gactgctgag ctctaccgta tatgtccatt cattcagcca |
| 1201 | agtactcttt acggaatgat gagtcaggtt cgtgagcatc cacgtcgtca actctaccgt |
| 1261 | gaatacctct cgcaacttcg ttcaacaatg atttctcgaa tggcaaagcc agaagaggtg |
| 1321 | ttgattgtgg aaaatgatca aggagaagtt gttcgtgaaa tggtcaaaga tactgattcg |
| 1381 | attgctttgt accgtaacat gcgtgaaacg cttgtctatt tgactcatct tgacaacaag |
| 1441 | gacactgaag tgaagatgac tgaaaagttg gcatcacaag tgaacggagg agagttttcg |
| 1501 | tggaagaatt tgaatcgttt gtgctgggct gttggttcga tttctggaac gatggttgaa |
| 1561 | gaagacgaaa agcgattcct tgttcttgtt attcgtgatt tactcgggct ctgtgaacag |
| 1621 | aaacgtggaa aggacaataa ggcagtgatt gcgtcaaaca tcatgtatgt tgtcggacag |
| 1681 | tacccgagat tccttcgagc tcactggaag ttcctgaaga cggttatcaa taagcttttc |
| 1741 | gagttcatgc acgagactca tgaaggtgta caggacatgg cttgtgatac attcatcaag |
| 1801 | atttccataa aatgtaagag gcatttcgtc atcgtccaac cagctgagaa taagcctttc |
| 1861 | gtcgaagaga tgctcgaaaa tttgactgga atcatctgcg atctttctca tgcacaagtt |
| 1921 | cacgtattct acgaggctgt tggacacatc atttctgcgc agatagatgg aaatctgcaa |
| 1981 | gaagacctga tcatgaagct tatggatatc ccaaatcgca catggaacga catcattgca |
| 2041 | gcagcatcca ctaacgacag tgtgctcgaa gagccggaga tggtcaaatc tgtcctgaat |
| 2101 | atactaaaaa caaatgtggc cgcctgcaaa tccattggat cttcgtttgt aacccaactc |
| 2161 | ggaaatatct acagtgatct tctatccctc tacaaaattc tatccgagaa ggtgtcccga |
| 2221 | gcagtgacaa ccgccggcga agaggctttg aagaatccat tggtaaagac gatgcgagct |
| 2281 | gtgaagcgag agattctcat ccttctatcg acattcattt caaagaacgg agatgccaag |
| 2341 | ctcattctgg acagtatagt tccaccattg ttcgatgcgg ttctcttcga ttaccagaag |
| 2401 | aatgtgccac aggcgagaga gccgaaagtg ttgtctttgc tcagcattct ggtcacacaa |
| 2461 | cttggatctc tcctctgtcc acaagtgccg agcatcctca gtgcggtttt ccaatgcagc |
| 2521 | attgacatga tcaacaagga tatggaagcg ttccccgagc atcgaaccaa cttcttcgag |
| 2581 | ctggtgcttt ctttggttca agagtgcttc ccggttttca tggaaatgcc tccagaggat |
| 2641 | cttggaacag tcatcgacgc cgtcgtttgg gcattccaac acacaatgcg caatgttgcg |
| 2701 | gaaattggtc tcgacattct caaagaatta ctggctcgtg tctcggagca ggatgacaag |
| 2761 | atcgctcaac cattctacaa gcgttactac attgatcttc tgaaacacgt gttagcagtt |
| 2821 | gcctgtgaca gttctcaagt tcatgtagcc ggtttgacct actacgccga agtgttgtgc |
| 2881 | gcgttattcc gtgctccaga gttttcgatc aaagttccgc tgaacgatgc gaatccttcg |
| 2941 | cagccgaata ttgattacat ttatgagcac atcggaggaa acttccaagc tcattttgat |
| 3001 | aatatgaacc aagatcaaat tcgcattatc atcaagggat tcttctcgtt caacactgaa |
| 3061 | atctccagta tgcgcaatca tctccgcgac tttttgattc aaatcaagga gcacaacgga |
| 3121 | gaagacacgt cggatttgta tcttgaagag cgagaagctg agattcaaca agctcaacag |
| 3181 | cgcaaacgag atgttcctgg aattctgaaa cctgacgagg tggaagatga ggatatgcgt |
| 3241 | TAA |

1. A method for treating a neurodegenerative disease in an individual comprising administering an inhibitor of exportin-1 (XPO1) or a fragment thereof to the individual.

2. (canceled)

3. The method of claim 1, wherein the neurodegenerative disease is selected from the group consisting of Alzheimer's disease, Amyotrophic lateral sclerosis (ALS), neurodegeneration in adult cases of Down's syndrome, Dementia puglistica, Pick's disease, Guam parkinsonism dementia complex, Fronto-temporal dementia, Cortico-Basal Degeneration, Pallido-Pontal-Nigral Degeneration, Progressive Nuclear Palsy, Parkinsonism of Chromosome 17 (FTDP-17), Parkinson's disease, Dementia with Lewy bodies, Huntington's disease, Multiple System Atrophy, fatty liver disease (liver steatosis), a1-anti-trypsin deficiency, muscle diseases, sporadic inclusion body myositis, limb girdle muscular dystrophy type 2B, and Miyoshi myopathy.

4. The method of claim 3, wherein the neurodegenerative disease comprises Alzheimer's disease.

5. The method of claim 4, wherein administration of the inhibitor of XPO1 or a fragment thereof results in increased clearance of Aβ42.

6. The method of claim 3, wherein the neurodegenerative disease comprises Huntington's disease.

7. The method of claim 6, wherein administration of the inhibitor of XPO1 or a fragment thereof results in decreased formation of Huntington's disease-like polyQ-containing protein aggregates.

8. The method of claim 7, wherein the polyQ-containing protein aggregate is Q35 or Q40.

9. The method of claim 1, wherein the individual has not been diagnosed with cancer.

10. The method of claim 1, wherein administration of the inhibitor of XPO1 or a fragment thereof results in the accumulation of autophagy-associated transcription factors in the nuclei of neurons and/or neural-related cells in the individual.

11. The method of claim 10, wherein the autophagy-associated transcription factor comprises Transcription factor EB (TFEB).

12. The method of claim 1, wherein administration of the inhibitor of XPO1 or a fragment thereof results in increased expression of a gene encoding one or more of TFEB, Sequestosome-1 protein SQSTM1 p62 (p62), Microtubule-associated proteins 1A/1B light chain 3A (LC3), Forkhead box protein O (FOXO) or Arylsulfatase A (ARSA) polypeptides in neurons and/or neural-related cells in the individual.

13. The method of claim 1, wherein administration of the inhibitor of XPO1 or a fragment thereof results in increased autophagic flux in neurons and/or neural-related cells in the individual.

14. The method of claim 1, wherein the inhibitor of XPO1 or a fragment thereof comprises one or more agents selected from the group consisting of a small molecule chemical compound, an antisense oligonucleotide, a siRNA, a non-antibody peptide, or an antibody or functional fragment thereof.

15. The method of claim 14, wherein the inhibitor of XPO1 comprises an siRNA.

16. The method of claim 14, wherein the small molecule chemical compound is an inhibitor of nuclear export.

17. The method of claim 16, wherein the inhibitor of nuclear export is selected from the group consisting of Selenixor (KPT-330), KPT-276, KPT-185, and KPT-335 (Verdinexor).

18. The method of claim 14, wherein the inhibitor of XPO1 or a fragment thereof comprises a therapeutically effective amount of Selenixor (KPT-330).

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. A method for increasing the longevity of a cell comprising contacting the cell with an inhibitor of exportin-1 (XPO1).

32. (canceled)

33. (canceled)

34. (canceled)

35. (canceled)

36. (canceled)

37. (canceled)

38. (canceled)

39. (canceled)

40. (canceled)

41. The method of claim 1, wherein the XPO1 inhibitor is a compound of the structural formula I: 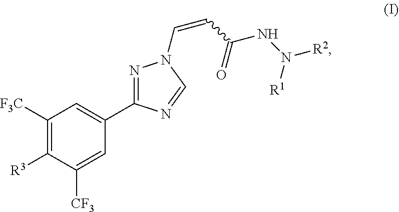
or a pharmaceutically acceptable salt thereof, wherein:
R¹ is selected from hydrogen and methyl;
R² is selected from pyridin-2-yl, pyridin-3-yl, pyridin-4-yl, pyrazin-2-yl, and quinoxalin-2-yl, pyrimidin-4-yl, 1,1-dioxotetrahydrothiophen-3-yl and cyclopropyl, wherein R is optionally substituted with one or more independent substituents selected from methyl and halogen; or
R¹ and R² are taken together with their intervening atoms to form 4-hydroxypiperidin-1-yl, pyrrolidin-1-yl, azepan-1-yl, 4-benzylpiperazin-1-yl, 4-ethylpiperazin-1-yl, 3-hydroxyazetidin-1-yl, or morpholin-4-yl;
R³ is selected from hydrogen and halo; and [symbol]
represents a single bond wherein a carbon-carbon double bond bound thereto is in an (E)- or (Z)-configuration.

[symbol]

[symbol]

42. The method of claim 41, wherein the compound is Selinexor (KPT-330):

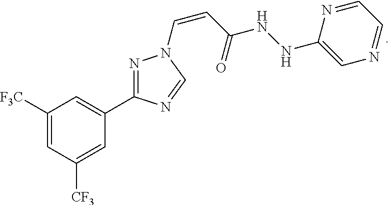 [SMILES: O=C(/C=C\n1cnc(-c2cc(C(F)(F)F)cc(C(F)(F)F)c2)n1)NNc1cnccn1]

43. The method of claim 41, wherein the compound is Verdinexor (KPT-335):

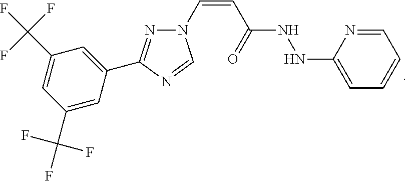 [SMILES: O=C(/C=C\n1cnc(-c2cc(C(F)(F)F)cc(C(F)(F)F)c2)n1)NNc1ccccn1]

44. (canceled)

45. (canceled)

46. (canceled)

47. The method of claim 1, wherein the XPO1 inhibitor is a compound of the structural formula II: 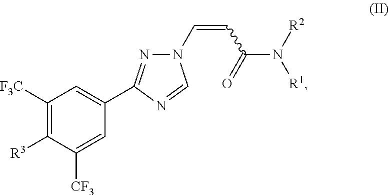
or a pharmaceutically acceptable salt thereof, wherein:
R¹ is selected from hydrogen and methyl;
R² is selected from pyridin-2-yl, pyridin-3-yl, pyridin-4-yl, pyrazin-2-yl, and quinoxalin-2-yl, pyrimidin-4-yl, 1,1-dioxotetrahydrothiophen-3-yl and cyclopropyl, wherein R is optionally substituted with one or more independent substituents selected from methyl and halogen; or
R¹ and R² are taken together with their intervening atoms to form 4-hydroxypiperidin-1-yl, pyrrolidin-1-yl, azepan-1-yl, 4-benzylpiperazin-1-yl, 4-ethylpiperazin-1-yl, 3-hydroxyazetidin-1-yl, azetidin-1-yl, or morpholin-4-yl, optionally substituted with 1, 2, 3, or 4 fluorines;
R³ is selected from hydrogen and halo; and [symbol]
represents a single bond wherein a carbon-carbon double bond bound thereto is in an (E)- or (Z)-configuration.

48. The method of claim 41, wherein the compound is KPT-276:

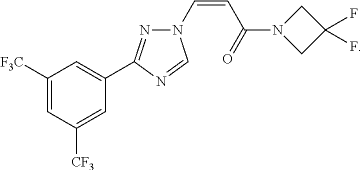 [SMILES: O=C(/C=C\n1cnc(-c2cc(C(F)(F)F)cc(C(F)(F)F)c2)n1)N1CC(F)(F)C1]

49. (canceled)

50. (canceled)

51. The method of claim 1, wherein the XPO1 inhibitor is a compound of the structural formula III: 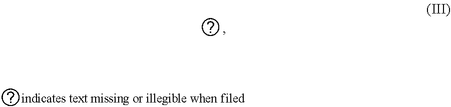
or a pharmaceutically acceptable salt thereof, wherein:
R¹ is an alkyl group having from 1 to 6 carbons;
R³ is selected from hydrogen and halo; and [symbol]
represents a single bond wherein a carbon-carbon double bond bound thereto is in an (E)- or (Z)-configuration.

52. The method of claim 51, wherein the compound is KPT-185:

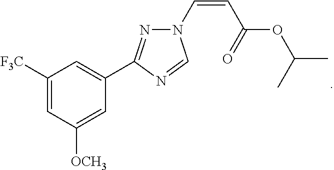 [SMILES: COc1cc(-c2ncn(/C=C\C(=O)OC(C)C)n2)cc(C(F)(F)F)c1]

53. (canceled)

54. (canceled)

55. (canceled)

56. (canceled)

57. (canceled)

58. (canceled)


SEQUENCE LISTING (32 sequences)

SEQ ID NO: 1 - protein, 1071 aa, Homo sapiens
MPAIMTMLADHAARQLLDFSQKLDINLLDNVVNCLYHGEGAQQRMAQEVLTHLKEHPDAWTRVDTILEFSQNMNTKYYGLQILENVIKTRWKILPRNQCEGIKKYVVGLIIKTSSDPTCVEKEKVYIGKLNMILVQILKQEWPKHWPTFISDIVGASRTSESLCQNNMVILKLLSEEVFDFSSGQITQVKSKHLKDSMCNEFSQIFQLCQFVMENSQNAPLVHATLETLLRFLNWIPLGYIFETKLISTLIYKFLNVPMFRNVSLKCLTEIAGVSVSQYEEQFVTLFTLTMMQLKQMLPLNTNIRLAYSNGKDDEQNFIQNLSLFLCTFLKEHDQLIEKRLNLRETLMEALHYMLLVSEVEETEIFKICLEYWNHLAAELYRESPFSTSASPLLSGSQHFDVPPRRQLYLPMLFKVRLLMVSRMAKPEEVLVVENDQGEVVREFMKDTDSINLYKNMRETLVYLTHLDYVDTERIMTEKLHNQVNGTEWSWKNLNTLCWAIGSISGAMHEEDEKRFLVTVIKDLLGLCEQKRGKDNKAIIASNIMYIVGQYPRFLRAHWKFLKTVVNKLFEFMHETHDGVQDMACDTFIKIAQKCRRHFVQVQVGEVMPFIDEILNNINTIICDLQPQQVHTFYEAVGYMIGAQTDQTVQEHLIEKYMLLPNQVWDSIIQQATKNVDILKDPETVKQLGSILKTNVRACKAVGHPFVIQLGRIYLDMLNVYKCLSENISAAIQANGEMVTKQPLIRSMRTVKRETLKLISGWVSRSNDPQMVAENFVPPLLDAVLIDYQRNVPAAREPEVLSTMAIIVNKLGGHITAEIPQIFDAVFECTLNMINKDFEEYPEHRTNFFLLLQAVNSHCFPAFLAIPPTQFKLVLDSIIWAFKHTMRNVADTGLQILFTLLQNVAQEEAAAQSFYQTYFCDILQHIFSVVTDTSHTAGLTMHASILAYMFNLVEEGKISTSLNPGNPVNNQIFLQEYVANLLKSAFPHLQDAQVKLFVTGLFSLNQDIPAFKEHLRDFLVQIKEFAGEDTSDLFLEEREIALRQADEEKHKRQMSVPGIFNPHEIPEEMCD

SEQ ID NO: 2 - DNA, 4890 bp, Homo sapiens
aggaaggccacgtccccggggagggacgcccgactcgatggtctgcgcagggcccgtcggcaaccggttccgagtttgaggcactaggaggagggggagaagcggctgcagcggccgcggcaggagcagcgggagctacagcatcagcaagagcaacagtagctacagccccggcggcggtgcctgttccagtctttgctgctgcagtccgtgcaaccacccagagggggaggggggaaccaccagtcgctgaggaacaagagaaggggggaaagtttaggcgagccttgggggggggggggccagcgccggagccgcgtgagagagggagccgtgttttggtaggggggagtcggactgcaactggcagcagagcgtctccccggccgtgtggactctacaccccctactcctgccgcttctgctgctgcctgtggctggagggtccccctggggctgaatctttgggacttgaccccgttccctcccccttccctcactccccagccgggcgggagcatttattccccagattaattccccttttgggggggggcgggtgtgtgtgtgtgtgtgtgtgtgtgtgtgtgttgggggaagcgtccctgaaatagtaaatattattgagctctttttgcccttttcctgtccgtttttttaatttccttttttgaggtgggaaaactgaaacccaccttgattcgtcccctctcccccctccccaccttccctcgccctaatcccccaacgaggaaggaaggagcagttggttcaatctctggtaatctatgccagcaattatgacaatgttagcagaccatgcagctcgtcagctgcttgatttcagccaaaaactggatatcaacttattagataatgtggtgaattgcttataccatggagaaggagcccagcaaagaatggctcaagaagtactgacacatttaaaggagcatcctgatgcttggacaagagtcgacacaattttggaattttctcagaatatgaatacgaaatactatggactacaaattttggaaaatgtgataaaaacaaggtggaagattcttccaaggaaccagtgcgaaggaataaaaaaatacgttgttggcctcattatcaagacgtcatctgacccaacttgtgtagagaaagaaaaggtgtatatcggaaaattaaatatgatccttgttcagatactgaaacaagaatggcccaaacattggccaacttttatcagtgatattgttggagcaagtaggaccagcgaaagtctctgtcaaaataatatggtgattcttaaactcttgagtgaagaagtatttgatttctctagtggacagataacccaagtcaaatctaagcatttaaaagacagcatgtgcaatgaattctcacagatatttcaactgtgtcagtttgtaatggaaaattctcaaaatgctccacttgtacatgcaaccttggaaacattgctcagatttctgaactggattcccctgggatatatttttgagaccaaattaatcagcacattgatttataagttcctgaatgttccaatgtttcgaaatgtctctctgaagtgcctcactgagattgctggtgtgagtgtaagccaatatgaagaacaatttgtaacactatttactctgacaatgatgcaactaaagcagatgcttcctttaaataccaatattcgacttgcgtactcaaatggaaaagatgatgaacagaacttcattcaaaatctcagtttgtttctctgcacctttcttaaggaacatgatcaacttatagaaaaaagattaaatctcagggaaactcttatggaggcccttcattatatgttgttggtatctgaagtagaagaaactgaaatctttaaaatttgtcttgaatactggaatcatttggctgctgaactctatagagagagtccattctctacatctgcctctccgttgctttctggaagtcaacattttgatgttcctcccaggagacagctatatttgcccatgttattcaaggtccgtttattaatggttagtcgaatggctaaaccagaggaagtattggttgtagagaatgatcaaggagaagttgtgagagaattcatgaaggatacagattccataaatttgtataagaatatgagggaaacattggtttatcttactcatctggattatgtagatacagaaagaataatgacagagaagcttcacaatcaagtgaatggtacagagtggtcatggaaaaatttgaatacattgtgttgggcaataggctccattagtggagcaatgcatgaagaggacgaaaaacgatttcttgttactgttataaaggatctattaggattatgtgaacagaaaagaggcaaagataataaagctattattgcatcaaatatcatgtacatagtaggtcaatacccacgttttttgagagctcactggaaatttctgaagactgtagttaacaagctgttcgaattcatgcatgagacccatgatggagtccaggatatggcttgtgatactttcattaaaatagcccaaaaatgccgcaggcatttcgttcaggttcaggttggagaagtgatgccatttattgatgaaattttgaacaacattaacactattatttgtgatcttcagcctcaacaggttcatacgttttatgaagctgtggggtacatgattggtgcacaaacagatcaaacagtacaagaacacttgatagaaaagtacatgttactccctaatcaagtgtgggatagtataatccagcaggcaaccaaaaatgtggatatactgaaagatcctgaaacagtcaagcagcttggtagcattttgaaaacaaatgtgagagcctgcaaagctgttggacacccctttgtaattcagcttggaagaatttatttagatatgcttaatgtatacaagtgcctcagtgaaaatatttctgcagctatccaagctaatggtgaaatggttacaaagcaaccattgattagaagtatgcgaactgtaaaaagggaaactttaaagttaatatctggttgggtgagccgatccaatgatccacagatggtcgctgaaaattttgttccccctctgttggatgcagttctcattgattatcagagaaatgtcccagctgctagagaaccagaagtgcttagtactatggccataattgtcaacaagttagggggacatataacagctgaaatacctcaaatatttgatgctgtttttgaatgcacattgaatatgataaataaggactttgaagaatatcctgaacatagaacgaactttttcttactacttcaggctgtcaattctcattgtttcccagcattccttgctattccacctacacagtttaaacttgttttggattccatcatttgggctttcaaacatactatgaggaatgtcgcagatacgggcttacagatactttttacactcttacaaaatgttgcacaagaagaagctgcagctcagagtttttatcaaacttatttttgtgatattctccagcatatcttttctgttgtgacagacacttcacatactgctggtttaacaatgcatgcatcaattcttgcatatatgtttaatttggttgaagaaggaaaaataagtacatcattaaatcctggaaatccagttaacaaccaaatctttcttcaggaatatgtggctaatctccttaagtcggccttccctcacctacaagatgctcaagtaaagctctttgtgacagggcttttcagcttaaatcaagatattcctgctttcaaggaacatttaagagatttcctagttcaaataaaggaatttgcaggtgaagacacttctgatttgtttttggaagagagagaaatagccctacggcaggctgatgaagagaaacataaacgtcaaatgtctgtccctggcatctttaatccacatgagattccagaagaaatgtgtgattaaaatccaaattcatgctgttttttttctctgcaactcgttagcagaggaaaacagcatgtgggtatttgtcgaccaaaatgatgccaatttgtaaattaaaatgtcacctagtggccctttttcttatgtgtttttttgtataagaaattttctgtgaaatatccttccattgtttaagcttttgttttggtcatctttatttagtttgcatgaagttgaaaattaaggcatttttaaaaattttacttcatgcccatttttgtggctgggctggggggaggaggcaaattcgatttgaacatatacttgtaattctaatgcaaaattatacaatttttcctgtaaacaataccaatttttaattagggagcattttccttctagtctatttcagcctagaagaaaagataatgagtaaaacaaattgcgttgtttaaaggattatagtgctgcattgtctgaagttagcacctcttggactgaatcgtttgtctagactacatgtattacaaagtctctttggcaagattgcagcaagatcatgtgcatatcatcccattgtaaagcgacttcaaaaatatgggaacacagttagttatttttacacagttctttttgtttttgtgtgtgtgtgctgtcgcttgtcgacaacagctttttgttttcctcaatgaggagtgttgctcatttgtgagccttcattaactcgaagtgaaatggttaaaaatatttatcctgttagaataggctgcatctttttaacaactcattaaaaaacaaaacaactctggcttttgagatgacttatactaatttacattgtttaccaagctgtagtgctttaagaacactacttaaaaagcaaaataaacttggtttacattta

SEQ ID NO: 3 - protein, 1080 aa, Caenorhabditis elegans
MAVSAMEVLSEAKRQFAQGDRIDVTLLDQVVEIMNRMSGKEQAEANQILMSLKEERDSWTKVDAILQYSQLNESKYFALQILETVIQHKWKSLPQVQREGIKSYIITKMFELSSDQSVMEQSQLLLHKLNLVLVQIVKQDWPKAWPTFITDIVDSSKNNETVCINNMNILSLLSEEVFDFGSQNLTQAKEQHLKQQFCGQFQEVFTLCVSILEKCPSNSMVQATLKTLQRFLTWIPVGYVFETNITELLSENFLSLEVYRVIALQCLTEISQIQVETNDPSYDEKLVKMFCSTMRHISQVLSLDLDLAAVYKDASDQDQKLISSLAQFLVAFIKEHVHLIEVTDEPLTEAKILMRESHDYAIQLLLKITLIEEMEVFKVCLDCWCWLTAELYRICPFIQPSTLYGMMSQVREHPRRQLYREYLSQLRSTMISRMAKPEEVLIVENDQGEVVREMVKDTDSIALYRNMRETLVYLTHLDNKDTEVKMTEKLASQVNGGEFSWKNLNRLCWAVGSISGTMVEEDEKRFLVLVIRDLLGLCEQKRGKDNKAVIASNIMYVVGQYPRFLRAHWKFLKTVINKLFEFMHETHEGVQDMACDTFIKISIKCKRHFVIVQPAENKPFVEEMLENLTGIICDLSHAQVHVFYEAVGHIISAQIDGNLQEDLIMKLMDIPNRTWNDIIAAASTNDSVLEEPEMVKSVLNILKTNVAACKSIGSSFVTQLGNIYSDLLSLYKILSEKVSRAVTTAGEEALKNPLVKTMRAVKREILILLSTFISKNGDAKLILDSIVPPLFDAVLFDYQKNVPQAREPKVLSLLSILVTQLGSLLCPQVPSILSAVFQCSIDMINKDMEAFPEHRTNFFELVLSLVQECFPVFMEMPPEDLGTVIDAVVWAFQHTMRNVAEIGLDILKELLARVSEQDDKIAQPFYKRYYIDLLKHVLAVACDSSQVHVAGLTYYAEVLCALFRAPEFSIKVPLNDANPSQPNIDYIYEHIGGNFQAHFDNMNQDQIRIIIKGFFSFNTEISSMRNHLRDFLIQIKEHNGEDTSDLYLEEREAEIQQAQQRKRDVPGILKPDEVEDEDMR

SEQ ID NO: 4 - DNA, 3243 bp, Caenorhabditis elegans
atggctgtctcagcaatggaagtgctctctgaagcgaagcgtcaattcgcccaaggtgaccgtatcgatgtgactcttttggatcaagtcgtcgagattatgaatcgaatgagcggaaaagaacaggccgaggctaaccaaatcctcatgtcgctcaaggaagaacgcgattcgtggaccaaagtcgatgcgattcttcaatattcgcagctaaatgagtccaagtattttgcacttcaaatccttgagactgtcatccaacacaaatggaagtcgttgccacaagtccaacgtgaaggaatcaagtcgtacatcatcaccaaaatgttcgaattgtcttctgatcagagtgtcatggaacaaagtcaactgcttcttcacaagttgaacctcgttttggttcaaattgtcaaacaagattggccaaaggcatggccaacattcatcaccgatattgttgattcatcgaagaacaacgagaccgtttgcatcaacaatatgaatattctgagcttgttgagcgaagaagtatttgatttcggatcccaaaacctcacccaagccaaggaacaacatctgaaacaacaattctgtggacagttccaagaagtattcacactgtgtgtcagcattctcgagaaatgtccatccaactctatggttcaagctaccttgaagactcttcaacgattcctcacctggattccagttggctacgttttcgagacaaacatcacggaattgctgtctgaaaacttcctttcgcttgaagtgtatcgtgtcatcgctcttcaatgtctcacggaaatttcacaaattcaagttgaaaccaacgatcccagctacgacgaaaagcttgtcaaaatgttctgctctacaatgcgccacattagccaagttctatctttggacctcgacctggctgctgtctacaaagatgcttccgatcaggatcagaaactcatcagtagtctggctcaatttcttgtcgcgttcatcaaagagcacgttcatctgattgaagtaactgatgagccattaactgaggctaaaattttaatgcgagaatctcacgactatgctattcaacttcttttgaaaatcactttgatcgaagaaatggaggttttcaaagtttgcctcgattgttggtgctggttgactgctgagctctaccgtatatgtccattcattcagccaagtactctttacggaatgatgagtcaggttcgtgagcatccacgtcgtcaactctaccgtgaatacctctcgcaacttcgttcaacaatgatttctcgaatggcaaagccagaagaggtgttgattgtggaaaatgatcaaggagaagttgttcgtgaaatggtcaaagatactgattcgattgctttgtaccgtaacatgcgtgaaacgcttgtctatttgactcatcttgacaacaaggacactgaagtgaagatgactgaaaagttggcatcacaagtgaacggaggagagttttcgtggaagaatttgaatcgtttgtgctgggctgttggttcgatttctggaacgatggttgaagaagacgaaaagcgattccttgttcttgttattcgtgatttactcgggctctgtgaacagaaacgtggaaaggacaataaggcagtgattgcgtcaaacatcatgtatgttgtcggacagtacccgagattccttcgagctcactggaagttcctgaagacggttatcaataagcttttcgagttcatgcacgagactcatgaaggtgtacaggacatggcttgtgatacattcatcaagatttccataaaatgtaagaggcatttcgtcatcgtccaaccagctgagaataagcctttcgtcgaagagatgctcgaaaatttgactggaatcatctgcgatctttctcatgcacaagttcacgtattctacgaggctgttggacacatcatttctgcgcagatagatggaaatctgcaagaagacctgatcatgaagcttatggatatcccaaatcgcacatggaacgacatcattgcagcagcatccactaacgacagtgtgctcgaagagccggagatggtcaaatctgtcctgaatatactaaaaacaaatgtggccgcctgcaaatccattggatcttcgtttgtaacccaactcggaaatatctacagtgatcttctatccctctacaaaattctatccgagaaggtgtcccgagcagtgacaaccgccggcgaagaggctttgaagaatccattggtaaagacgatgcgagctgtgaagcgagagattctcatccttctatcgacattcatttcaaagaacggagatgccaagctcattctggacagtatagttccaccattgttcgatgcggttctcttcgattaccagaagaatgtgccacaggcgagagagccgaaagtgttgtctttgctcagcattctggtcacacaacttggatctctcctctgtccacaagtgccgagcatcctcagtgcggttttccaatgcagcattgacatgatcaacaaggatatggaagcgttccccgagcatcgaaccaacttcttcgagctggtgctttctttggttcaagagtgcttcccggttttcatggaaatgcctccagaggatcttggaacagtcatcgacgccgtcgtttgggcattccaacacacaatgcgcaatgttgcggaaattggtctcgacattctcaaagaattactggctcgtgtctcggagcaggatgacaagatcgctcaaccattctacaagcgttactacattgatcttctgaaacacgtgttagcagttgcctgtgacagttctcaagttcatgtagccggtttgacctactacgccgaagtgttgtgcgcgttattccgtgctccagagttttcgatcaaagttccgctgaacgatgcgaatccttcgcagccgaatattgattacatttatgagcacatcggaggaaacttccaagctcattttgataatatgaaccaagatcaaattcgcattatcatcaagggattcttctcgttcaacactgaaatctccagtatgcgcaatcatctccgcgactttttgattcaaatcaaggagcacaacggagaagacacgtcggatttgtatcttgaagagcgagaagctgagattcaacaagctcaacagcgcaaacgagatgttcctggaattctgaaacctgacgaggtggaagatgaggatatgcgttaa

SEQ ID NO: 5 - DNA, 962 bp, Artificial Sequence
  RNAi sequence based on the cosmid sequence ZK742.1 of the C. elegans genome
  misc_feature (17)..(17): n is a, c, g, or t
cgcgcgtatacgactcnctatagggcgaattgggtaccgggccccccctcgaggtcgacggtatcgataagcttgattctcgtgcatgaactcgaaaagcttattgataaccgtcttcaggaacttccagtgagctcgaaggaatctcgggtactgtccgacaacatacatgatgtttgacgcaatcactgccttattgtcctttccacgtttctgttcacagagcccgagtaaatcacgaataacaagaacaaggaatcgcttttcgtcttcttcaaccatcgttccagaaatcgaaccaacagcccagcacaaacgattcaaattcttccacgaaaactctcctccgttcacttgtgatgccaacttttcagtcatcttcacttcagtgtccttgttgtcaagatgagtcaaatagacaagcgtttcacgcatgttacggtacaaagcaatcgaatcagtatctttgaccatttcacgaacaacttctccttgatcattttccacaatcaacacctcttctggctttgccattcgagaaatcattgttgaacgaagttgcgagaggtattcacggtagagttgacgacgtggatgctcacgaacctgactcatcattccgtaaagagtacttggctgaatgaatggacatatacggtagagctcagcagtcaaccagcaccaacaatcgaggcaaactttgaaaacctccatttcttcgatcaaagtgattttcaaaagaagttgaatagcatagtcgtgagattctcgcattaaaattttagcctcagttaatggctcatcagttacttcaatcagatgaacgtgctctttgatgaacgcgacaagaaattgagccagactactgatgagtttctgatcctgatcggaagcatctttgtagacagcagccaggtcgaggtccaaagatagaacttggctaatgtggcgcattgtagagc

SEQ ID NO: 6 - DNA, 23 bp, Artificial Sequence
  act-1 Forward primer
ctacgaacttcctgacggacaag

SEQ ID NO: 7 - DNA, 17 bp, Artificial Sequence
  act-1 Reverse primer
ccggcggactccatacc

SEQ ID NO: 8 - DNA, 20 bp, Artificial Sequence
  cyn-1 Forward primer
gtgtcaccatggagttgttc

SEQ ID NO: 9 - DNA, 20 bp, Artificial Sequence
  cyn-1 Reverse primer
tccgtagattgattcaccac

SEQ ID NO: 10 - DNA, 21 bp, Artificial Sequence
  cdc-42 Forward primer
ctgctggacaggaagattacg

SEQ ID NO: 11 - DNA, 21 bp, Artificial Sequence
  cdc-42 Reverse primer
ctcggacattctcgaatgaag

SEQ ID NO: 12 - DNA, 21 bp, Artificial Sequence
  pmp-3 Forward primer
gttcccgtgttcatcactcat

SEQ ID NO: 13 - DNA, 21 bp, Artificial Sequence
  pmp-3 Reverse primer
acaccgtcgagaagctgtaga

SEQ ID NO: 14 - DNA, 20 bp, Artificial Sequence
  xpo-1 Forward primer
aagaacaggccgaggctaac

SEQ ID NO: 15 - DNA, 20 bp, Artificial Sequence
  xpo-1 Reverse primer
gttggacttgtggcaacgac

SEQ ID NO: 16 - DNA, 20 bp, Artificial Sequence
  lgg-1 Forward primer
acccagaccgtattccagtg

SEQ ID NO: 17 - DNA, 20 bp, Artificial Sequence
  lgg-1 Reverse primer
acgaagttggatgcgttttc

SEQ ID NO: 18 - DNA, 20 bp, Artificial Sequence
  lgg-2 Forward primer
gcatataaccgttgccgagc

SEQ ID NO: 19 - DNA, 20 bp, Artificial Sequence
  lgg-2 Reverse primer
caaagccatctggatcacgc

SEQ ID NO: 20 - DNA, 20 bp, Artificial Sequence
  sgst-1 Forward primer
tggctgctgcatcatccgct

SEQ ID NO: 21 - DNA, 20 bp, Artificial Sequence
  sgst-1 Reverse primer
tcaatcgtgccgagaccggg

SEQ ID NO: 22 - DNA, 20 bp, Artificial Sequence
  hlh-30 Forward primer
ctcatcggccggcgctcatc

SEQ ID NO: 23 - DNA, 20 bp, Artificial Sequence
  hlh-30 Reverse primer
agaacgcgatgcgtggtggg

SEQ ID NO: 24 - DNA, 20 bp, Artificial Sequence
  lipl-1 Forward primer
tgcaacacggtcttgaatgc

SEQ ID NO: 25 - DNA, 20 bp, Artificial Sequence
  lipl-1 Reverse primer
ccaatcccagaatgccgagt

SEQ ID NO: 26 - DNA, 20 bp, Artificial Sequence
  lipl-2 Forward primer
gttgctagcatgtgccagtg

SEQ ID NO: 27 - DNA, 20 bp, Artificial Sequence
  lipl-2 Reverse primer
tgccagaaagcagtttccga

SEQ ID NO: 28 - DNA, 20 bp, Artificial Sequence
  lipl-3 Forward primer
cgatggggttatccggcaat

SEQ ID NO: 29 - DNA, 20 bp, Artificial Sequence
  lipl-3 Reverse primer
cgggcaggttcatagtccag

SEQ ID NO: 30 - DNA, 21 bp, Artificial Sequence
  lipl-4 Forward primer
acaggtattgcggatgtttcc

SEQ ID NO: 31 - DNA, 20 bp, Artificial Sequence
  lipl-4 Reverse primer
gcatttgttccccaaatgaa

SEQ ID NO: 32 - protein, 1001 aa, Artificial Sequence
  Drosophila melanogaster - Embargoed
MATMLTSDEAGKLLDFSQKLDINLLDKIVEVVYTAQGEQLRLAQSILTTLKEHPEAWTRVDSILEYSQNQRTKFYALQILEEVIKTRWKVLPRNQCEGIKKYVVSLIIKTSSDPIVMEQNKVYLNKLNMILVHILKREWPRNWETFISDIVGASKTNESLCMNNMVILKNLSEEVFDFSQGQITQTKAKHLKDTMCSEFSQIFTLCSFVLENSMNAALIHVTLETLLRFLNWIPLGYIFETQQIETLIFKFLSVPMFRNVTLKCLSEIAGLTAANYDENFATLFKDTMVQLEQIVGQNMNMNHVFKHGSDTEQELVLNLAMFLCTFLKEHGKLVEDAKYVDYLNQALMYLVMISEVEDVEVFKICLEYWNSLVEDLYNSEFFHPTLESTKRQQVYPRRRFYAPILSKVRFIMISRMAKPEEVLVVENENGEVVREFMKDTNSINLYKNMRETLVFLTHLDSVDTDRIMTLKLLNQVNGSEFSWKNLNTLCWAIGSISGAFCEEDEKRFLVTVIKDLLGLCIKIAIKCRRYFVTIQPNEACTFIDEILTTMSSIICDLQPQQVHTFYEAVGYMISAQVDQVQQDVLIERYMQLNQVWDDIISRASKNVDFLKNMTAVKQLGSILKTNVAACKALGHAYVIQLGRIYLDMLNVYKITSENIIQAIEVNGVNVNNQPLIKTMHVKKETLNLISEWVSRSNDNQLVMDNFIPPLLDAILLDYQRCKVPSAREPKVLSAMAIIVHKLRQHITNEVPKIFDAVFECTLDMINKNFEDFPQHRLSFYELLQAVNAHCFKAFLNIPPAQFKLVFDSVVWAFKHTMRNVADMGLNILFKMLQNLDQHPGAAQSFYQTYFTDILMQIFSVVTDTSHTAGLPNHAIILAYMFSLVENRKITVNLGPIPDNMIFIQEYVASLLKSAFTHLSDNQVKVFVTGLFNLDENVQAFKEHLRDFLIQIREATGEDDSDLYLEEREAALAEEQSNKHQMQRNIPGMLNPHELPEDMQDE